(19) United States
(12) Patent Application Publication
(10) Pub. No.: US 2025/0241597 A1
(43) Pub. Date: Jul. 31, 2025

(54) TECHNIQUES FOR NOISE REDUCTION

(71) Applicant: Oura Health Oy, Oulu (FI)

(72) Inventors: Jaakko Tapio Vartiainen, Oulu (FI); Jukka Tapani Mäkinen, Oulu (FI); Kirsi Marja Maansaari, Oulu (FI); Olli Petteri Heikkinen, Oulu (FI); Mika Petteri Kangas, Oulu (FI); Pekka Johannes Karhula, Oulu (FI)

(21) Appl. No.: 18/422,985

(22) Filed: Jan. 25, 2024

(51) Int. Cl. A61B 5/00; A61B 5/1455; G16H 40/67

(52) U.S. Cl. CPC A61B 5/7214; A61B 5/14552; A61B 5/6826; A61B 5/721; A61B 5/7246; A61B 5/725; A61B 5/726; G16H 40/67

(57) ABSTRACT

Methods, systems, and devices for noise filtering for a wearable ring device are described. The noise filtering procedure may employ one or more mathematical techniques or algorithms aimed at improving the quality of signals acquired by the wearable ring device. The device may measure a first signal including physiological phenomenon and a noise component. The first signal may undergo a discrete wavelet transform (DWT), where the input signal may be decomposed into various sets of coefficients, each set of coefficients representing a specific frequency range. The DWT may filter out noise by eliminating higher frequencies, as well as through a noise thresholding mechanism. The first signal may also undergo an independent component analysis (ICA), where the first signal is decomposed into various independent components. The ICA may filter out noise by identifying which ICA components correlate with the accelerometer measurements. The device may calculate a clean signal based on filtering out the noise.

### FIELD OF TECHNOLOGY

The following relates to wearable devices and data processing, including techniques for noise reduction.

### BACKGROUND

Some wearable devices may be configured to collect data from users associated with physiological phenomenon, motion artifacts, or both, such as heart rate, oxygen saturation level, blood pressure, or the like. However, movement of the user may result in noise within collected physiological measurements. As such, there are technical challenges with removing noise from signals acquired via wearable devices, leading to inaccurate physiological data.

### BRIEF DESCRIPTION OF THE DRAWINGS

FIG. 1 illustrates an example of a system that supports techniques for noise reduction in accordance with aspects of the present disclosure.

FIG. 2 illustrates an example of a system that supports techniques for noise reduction in accordance with aspects of the present disclosure.

FIG. 3 shows an example of a noise filtering procedure that supports techniques for noise reduction in accordance with aspects of the present disclosure.

FIG. 4 shows an example of a noise filtering procedure that supports techniques for noise reduction in accordance with aspects of the present disclosure.

FIG. 5 shows a block diagram of an apparatus that supports techniques for noise reduction in accordance with aspects of the present disclosure.

FIG. 6 shows a block diagram of a wearable device manager that supports techniques for noise reduction in accordance with aspects of the present disclosure.

FIG. 7 shows a diagram of a system including a device that supports techniques for noise reduction in accordance with aspects of the present disclosure.

FIGS. 8 and 9 show flowcharts illustrating methods that support techniques for noise reduction in accordance with aspects of the present disclosure.

### DETAILED DESCRIPTION

Some wearable devices may be configured to collect data from users associated with movement and other activities. For example, some wearable devices may be configured to continuously acquire physiological data associated with a user including temperature data, blood pressure data, heart rate data, and the like. In order to efficiently and accurately track physiological data, a wearable device may be configured to collect data continuously while the user wears the device.

Measurements from different sensors of a wearable device may vary. Particularly, physiological data, motion data, or both, obtained by the wearable device may be susceptible to distortions originating from the inherent variability of these sensors. There may be different types of noise, such as hardware noise, physiological noise, and environmental noise. Hardware noise may include noise that is attributable to sensors used to acquire physiological data. Comparatively, physiological noise may be attributable to physiological characteristics/features of a user's tissue (e.g., different layers of tissue, bone, reflections from blood vessels, etc.), that may cause noise within acquired physiological measurements (e.g., light reflecting from a blood vessel). Environmental noise may include noise attributable to an environment of the user, such as movement of the user, external light that interferes with light-based measurements, and the like. For instance, motion of a user may result in “motion artifacts” within collected physiological measurements/signals. Such motion artifacts may come from surface effects, where the optical interface between sensor and skin changes with the motion (e.g., contact/no-contact) or from physiological effects induced by motion. For instance, when a user moves their hand from a raised position to downward-pointing position, the hydrostatic pressure in the fingers changes and causes changes (e.g., motion artifacts/noise) in the PPG signal direct-current (DC)-level (likely also in the alternating current (AC)-levels).

In some cases, characteristics of the user's environment may manifest in forms of physiological-based noise. For instance, lower/higher air pressure (e.g., environmental characteristics) may cause changes in a user's internal body hydrostatic pressure that show up as signal DC-level changes/noise. In this case, the root cause of the noise is an environmental factor (e.g., air pressure), but it shows up in the measurement as a physiological feature (increased/decreased hydrostatic pressure).

The presence of these diverse sources of noise may cause inconsistencies in the physiological data, motion data, or both. Sensors of the wearable device may be calibrated to ensure accurate acquisition of physiological data, mobility data, or both. However, the calibration process may not always effectively filter certain noise components from others, potentially resulting in improper sensor calibration. This deficiency in calibration may subsequently yield inaccurate or deceptive physiological data, motion data, or both, which may unfavorably impact the user's experience with the wearable device.

Accordingly, techniques described herein are directed to devices, methods and systems for implementing algorithmic techniques for noise filtering to improve the accuracy of signals obtained from sensors of a wearable device that may correspond to measured physiological data, motion data, or both. Specifically, the noise filtering procedure may employ one or more mathematical (e.g., computational) techniques or algorithms aimed at improving the quality of the acquired signals.

For the purposes of the present disclosure, the terms “signal” and “noise” may be measurement-feature-dependent, in that the dividing line between a “signal” and “noise” may be dependent on the respective physiological measurement being performed. In other words, what is considered a “signal” for one feature (e.g., physiological feature/measurement) may be considered “noise” for another feature.

For example, as described previously herein, motion of a user may result in “motion artifacts” within collected PPG data. Such motion artifacts would be considered to be “noise” when performing heart rate measurements. Conversely, in order to measure movement without a separate electromechanical sensor component, a wearable device (and/or related device) may use the “motion artifact” portion of the PPG sensor signal (assuming there is a good correlation between the motion artifacts and acceleration). In this regard, the “motion artifacts” may be considered to be “noise” when performing heart rate measurements, but may be considered to be a “signal” when measuring movements of the user.

In some cases, an input signal collected via a wearable device may undergo a discrete wavelet transform (DWT). As part of applying the DWT, the input signal may be decomposed into various sets of coefficients, where each set of coefficients represents a specific frequency range. The respective “sets of coefficients” may additionally or alternatively be referred to as “components.” The DWT may filter out noise by eliminating higher frequencies (or otherwise filtering out frequencies outside of a preferred frequency range), as well as through a noise thresholding mechanism. Noise thresholding may occur when a signal, such as an anomaly, exhibits a frequency higher than a predefined noise threshold. A device (e.g., a wearable device, a corresponding user device, and/or a server) may accumulate one or more of these anomalies and construct a noise reference, which may often be encountered during sporadic movements (e.g., hand gesture).

In additional or alternative cases, a device may establish a noise reference by utilizing an accelerometer to gain a deeper understanding of photoplethysmography (PPG) motion. In this scenario, the device may obtain different signals (e.g., from distinct light emitting elements) that may undergo an independent component analysis (ICA). Through this analysis, various ICA components, such as independent PPG sources like movements, steps, and heart rate, may be extracted from the initial signals. In some instances, the signals may serve as inputs, and the ICA components may become the outputs. Once the device identifies which ICA components correlate with the accelerometer measurements, the device may construct a noise reference. For instance, the device may compare each respective ICA component with accelerometer data. The device may evaluate the strength of the correlation between these measurements. Hand motion and the step counter may exhibit a strong correlation with the accelerometer. The device may compare the different physiological measurements to ascertain a noise component based on establishing a noise reference and/or determining whether to filter, cancel, or otherwise remove one or more signals. Following this determination, the device may eliminate the noise component. Thus the device may effectively filter the noise from the prior measurements, which may result in a cleaner signal and more accurate physiological data.

Aspects of the disclosure are initially described in the context of systems supporting physiological data collection from users via wearable devices. Additional aspects of the disclosure are described in the context of noise filtering procedures. Aspects of the disclosure are further illustrated by and described with reference to apparatus diagrams, system diagrams, and flowcharts that relate to computational techniques for noise reduction.

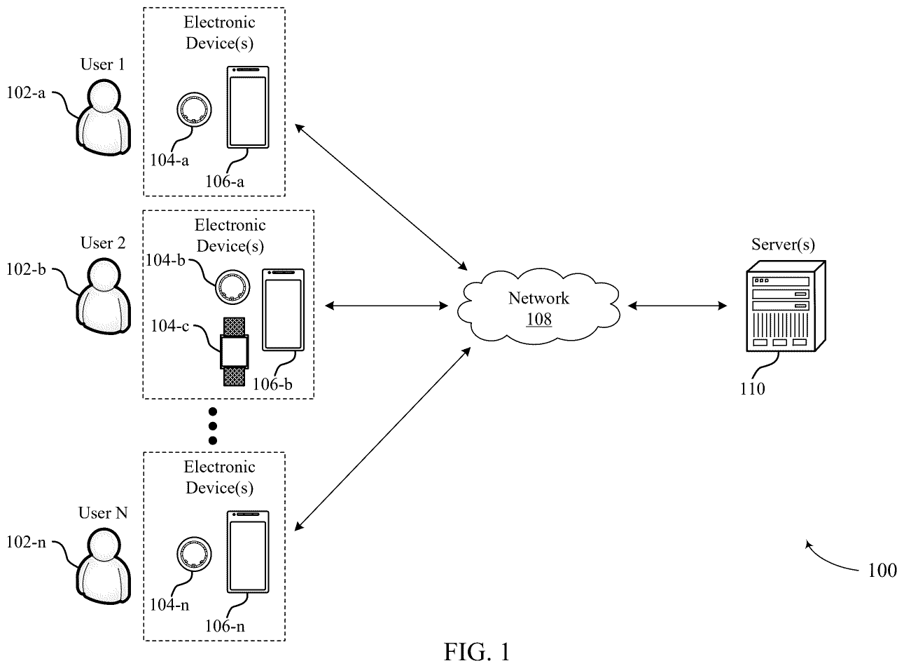

FIG. 1 illustrates an example of a system 100 that supports techniques for noise reduction in accordance with aspects of the present disclosure. The system 100 includes a plurality of electronic devices (e.g., wearable devices 104, user devices 106) that may be worn and/or operated by one or more users 102. The system 100 further includes a network 108 and one or more servers 110.

The electronic devices may include any electronic devices known in the art, including wearable devices 104 (e.g., ring wearable devices, watch wearable devices, etc.), user devices 106 (e.g., smartphones, laptops, tablets). The electronic devices associated with the respective users 102 may include one or more of the following functionalities: 1) measuring physiological data, 2) storing the measured data, 3) processing the data, 4) providing outputs (e.g., via GUIs) to a user 102 based on the processed data, and 5) communicating data with one another and/or other computing devices. Different electronic devices may perform one or more of the functionalities.

Example wearable devices 104 may include wearable computing devices, such as a ring computing device (hereinafter “ring”) configured to be worn on a user's 102 finger, a wrist computing device (e.g., a smart watch, fitness band, or bracelet) configured to be worn on a user's 102 wrist, and/or a head mounted computing device (e.g., glasses/goggles). Wearable devices 104 may also include bands, straps (e.g., flexible or inflexible bands or straps), stick-on sensors, and the like, that may be positioned in other locations, such as bands around the head (e.g., a forehead headband), arm (e.g., a forearm band and/or bicep band), and/or leg (e.g., a thigh or calf band), behind the ear, under the armpit, and the like. Wearable devices 104 may also be attached to, or included in, articles of clothing. For example, wearable devices 104 may be included in pockets and/or pouches on clothing. As another example, wearable device 104 may be clipped and/or pinned to clothing, or may otherwise be maintained within the vicinity of the user 102. Example articles of clothing may include, but are not limited to, hats, shirts, gloves, pants, socks, outerwear (e.g., jackets), and undergarments. In some implementations, wearable devices 104 may be included with other types of devices such as training/sporting devices that are used during physical activity. For example, wearable devices 104 may be attached to, or included in, a bicycle, skis, a tennis racket, a golf club, and/or training weights.

Much of the present disclosure may be described in the context of a ring wearable device 104. Accordingly, the terms “ring 104,” “wearable device 104,” and like terms, may be used interchangeably, unless noted otherwise herein. However, the use of the term “ring 104” is not to be regarded as limiting, as it is contemplated herein that aspects of the present disclosure may be performed using other wearable devices (e.g., watch wearable devices, necklace wearable device, bracelet wearable devices, earring wearable devices, anklet wearable devices, and the like).

In some aspects, user devices 106 may include handheld mobile computing devices, such as smartphones and tablet computing devices. User devices 106 may also include personal computers, such as laptop and desktop computing devices. Other example user devices 106 may include server computing devices that may communicate with other electronic devices (e.g., via the Internet). In some implementations, computing devices may include medical devices, such as external wearable computing devices (e.g., Holter monitors). Medical devices may also include implantable medical devices, such as pacemakers and cardioverter defibrillators. Other example user devices 106 may include home computing devices, such as internet of things (IoT) devices (e.g., IoT devices), smart televisions, smart speakers, smart displays (e.g., video call displays), hubs (e.g., wireless communication hubs), security systems, smart appliances (e.g., thermostats and refrigerators), and fitness equipment.

Some electronic devices (e.g., wearable devices 104, user devices 106) may measure physiological parameters of respective users 102, such as photoplethysmography waveforms, continuous skin temperature, a pulse waveform, respiration rate, heart rate, heart rate variability (HRV), actigraphy, galvanic skin response, pulse oximetry, blood oxygen saturation (SpO2), blood sugar levels (e.g., glucose metrics), and/or other physiological parameters. Some electronic devices that measure physiological parameters may also perform some/all of the calculations described herein. Some electronic devices may not measure physiological parameters, but may perform some/all of the calculations described herein. For example, a ring (e.g., wearable device 104), mobile device application, or a server computing device may process received physiological data that was measured by other devices.

In some implementations, a user 102 may operate, or may be associated with, multiple electronic devices, some of which may measure physiological parameters and some of which may process the measured physiological parameters. In some implementations, a user 102 may have a ring (e.g., wearable device 104) that measures physiological parameters. The user 102 may also have, or be associated with, a user device 106 (e.g., mobile device, smartphone), where the wearable device 104 and the user device 106 are communicatively coupled to one another. In some cases, the user device 106 may receive data from the wearable device 104 and perform some/all of the calculations described herein. In some implementations, the user device 106 may also measure physiological parameters described herein, such as motion/activity parameters.

For example, as illustrated in FIG. 1, a first user 102-a (User 1) may operate, or may be associated with, a wearable device 104-a (e.g., ring 104-a) and a user device 106-a that may operate as described herein. In this example, the user device 106-a associated with user 102-a may process/store physiological parameters measured by the ring 104-a. Comparatively, a second user 102-b (User 2) may be associated with a ring 104-b, a watch wearable device 104-c (e.g., watch 104-c), and a user device 106-b, where the user device 106-b associated with user 102-b may process/store physiological parameters measured by the ring 104-b and/or the watch 104-c. Moreover, an nth user 102-n (User N) may be associated with an arrangement of electronic devices described herein (e.g., ring 104-n, user device 106-n). In some aspects, wearable devices 104 (e.g., rings 104, watches 104) and other electronic devices may be communicatively coupled to the user devices 106 of the respective users 102 via Bluetooth, Wi-Fi, and other wireless protocols. Moreover, in some cases, the wearable device 104 and the user device 106 may be included within (or make up) the same device. For example, in some cases, the wearable device 104 may be configured to execute an application associated with the wearable device 104, and may be configured to display data via a GUI.

In some implementations, the rings 104 (e.g., wearable devices 104) of the system 100 may be configured to collect physiological data from the respective users 102 based on arterial blood flow within the user's finger. In particular, a ring 104 may utilize one or more light-emitting components, such as LEDs (e.g., red LEDs, green LEDs) that emit light on the palm-side of a user's finger to collect physiological data based on arterial blood flow within the user's finger. In general, the terms light-emitting components, light-emitting elements, and like terms, may include, but are not limited to, LEDs, micro LEDs, mini LEDs, laser diodes (LDs) (e.g., vertical cavity surface-emitting lasers (VCSELs), and the like.

In some cases, the system 100 may be configured to collect physiological data from the respective users 102 based on blood flow diffused into a microvascular bed of skin with capillaries and arterioles. For example, the system 100 may collect PPG data based on a measured amount of blood diffused into the microvascular system of capillaries and arterioles. In some implementations, the ring 104 may acquire the physiological data using a combination of both green and red LEDs. The physiological data may include any physiological data known in the art including, but not limited to, temperature data, accelerometer data (e.g., movement/motion data), heart rate data, HRV data, blood oxygen level data, or any combination thereof.

The use of both green and red LEDs may provide several advantages over other solutions, as red and green LEDs have been found to have their own distinct advantages when acquiring physiological data under different conditions (e.g., light/dark, active/inactive) and via different parts of the body, and the like. For example, green LEDs have been found to exhibit better performance during exercise. Moreover, using multiple LEDs (e.g., green and red LEDs) distributed around the ring 104 has been found to exhibit superior performance as compared to wearable devices that utilize LEDs that are positioned close to one another, such as within a watch wearable device. Furthermore, the blood vessels in the finger (e.g., arteries, capillaries) are more accessible via LEDs as compared to blood vessels in the wrist. In particular, arteries in the wrist are positioned on the bottom of the wrist (e.g., palm-side of the wrist), meaning only capillaries are accessible on the top of the wrist (e.g., back of hand side of the wrist), where wearable watch devices and similar devices are typically worn. As such, utilizing LEDs and other sensors within a ring 104 has been found to exhibit superior performance as compared to wearable devices worn on the wrist, as the ring 104 may have greater access to arteries (as compared to capillaries), thereby resulting in stronger signals and more valuable physiological data.

The electronic devices of the system 100 (e.g., user devices 106, wearable devices 104) may be communicatively coupled to one or more servers 110 via wired or wireless communication protocols. For example, as shown in FIG. 1, the electronic devices (e.g., user devices 106) may be communicatively coupled to one or more servers 110 via a network 108. The network 108 may implement transfer control protocol and internet protocol (TCP/IP), such as the Internet, or may implement other network 108 protocols. Network connections between the network 108 and the respective electronic devices may facilitate transport of data via email, web, text messages, mail, or any other appropriate form of interaction within a computer network 108. For example, in some implementations, the ring 104-a associated with the first user 102-a may be communicatively coupled to the user device 106-a, where the user device 106-a is communicatively coupled to the servers 110 via the network 108. In additional or alternative cases, wearable devices 104 (e.g., rings 104, watches 104) may be directly communicatively coupled to the network 108.

The system 100 may offer an on-demand database service between the user devices 106 and the one or more servers 110. In some cases, the servers 110 may receive data from the user devices 106 via the network 108, and may store and analyze the data. Similarly, the servers 110 may provide data to the user devices 106 via the network 108. In some cases, the servers 110 may be located at one or more data centers. The servers 110 may be used for data storage, management, and processing. In some implementations, the servers 110 may provide a web-based interface to the user device 106 via web browsers.

In some aspects, the system 100 may detect periods of time that a user 102 is asleep, and classify periods of time that the user 102 is asleep into one or more sleep stages (e.g., sleep stage classification). For example, as shown in FIG. 1, User 102-a may be associated with a wearable device 104-a (e.g., ring 104-a) and a user device 106-a. In this example, the ring 104-a may collect physiological data associated with the user 102-a, including temperature, heart rate, HRV, respiratory rate, and the like. In some aspects, data collected by the ring 104-a may be input to a machine learning classifier, where the machine learning classifier is configured to determine periods of time that the user 102-a is (or was) asleep. Moreover, the machine learning classifier may be configured to classify periods of time into different sleep stages, including an awake sleep stage, a rapid eye movement (REM) sleep stage, a light sleep stage (non-REM (NREM)), and a deep sleep stage (NREM). In some aspects, the classified sleep stages may be displayed to the user 102-a via a GUI of the user device 106-a. Sleep stage classification may be used to provide feedback to a user 102-a regarding the user's sleeping patterns, such as recommended bedtimes, recommended wake-up times, and the like. Moreover, in some implementations, sleep stage classification techniques described herein may be used to calculate scores for the respective user, such as Sleep Scores, Readiness Scores, and the like.

In some aspects, the system 100 may utilize circadian rhythm-derived features to further improve physiological data collection, data processing procedures, and other techniques described herein. The term circadian rhythm may refer to a natural, internal process that regulates an individual's sleep-wake cycle, that repeats approximately every 24 hours. In this regard, techniques described herein may utilize circadian rhythm adjustment models to improve physiological data collection, analysis, and data processing. For example, a circadian rhythm adjustment model may be input into a machine learning classifier along with physiological data collected from the user 102-a via the wearable device 104-a. In this example, the circadian rhythm adjustment model may be configured to “weight,” or adjust, physiological data collected throughout a user's natural, approximately 24-hour circadian rhythm. In some implementations, the system may initially start with a “baseline” circadian rhythm adjustment model, and may modify the baseline model using physiological data collected from each user 102 to generate tailored, individualized circadian rhythm adjustment models that are specific to each respective user 102.

In some aspects, the system 100 may utilize other biological rhythms to further improve physiological data collection, analysis, and processing by phase of these other rhythms. For example, if a weekly rhythm is detected within an individual's baseline data, then the model may be configured to adjust “weights” of data by day of the week. Biological rhythms that may require adjustment to the model by this method include: 1) ultradian (faster than a day rhythms, including sleep cycles in a sleep state, and oscillations from less than an hour to several hours periodicity in the measured physiological variables during wake state; 2) circadian rhythms; 3) non-endogenous daily rhythms shown to be imposed on top of circadian rhythms, as in work schedules; 4) weekly rhythms, or other artificial time periodicities exogenously imposed (e.g. in a hypothetical culture with 12 day “weeks,” 12 day rhythms could be used); 5) multi-day ovarian rhythms in women and spermatogenesis rhythms in men; 6) lunar rhythms (relevant for individuals living with low or no artificial lights); and 7) seasonal rhythms.

The biological rhythms are not always stationary rhythms. For example, many women experience variability in ovarian cycle length across cycles, and ultradian rhythms are not expected to occur at exactly the same time or periodicity across days even within a user. As such, signal processing techniques sufficient to quantify the frequency composition while preserving temporal resolution of these rhythms in physiological data may be used to improve detection of these rhythms, to assign phase of each rhythm to each moment in time measured, and to thereby modify adjustment models and comparisons of time intervals. The biological rhythm-adjustment models and parameters can be added in linear or non-linear combinations as appropriate to more accurately capture the dynamic physiological baselines of an individual or group of individuals.

In some aspects, the respective devices of the system 100 may support techniques for employing computation and mathematical techniques to filter noise from signals/measurements associated with one or more physiological phenomenon of a wearer (e.g., a user 102) of a wearable device 104. For example, physiological data, motion/mobility measurements, or both, collected using a wearable device 104 may be subject to noise from the variability in the different sensors. The noise filtering procedure may employ one or more mathematical (e.g., computational) techniques or algorithms aimed at improving the quality of the signals acquired by the wearable device 104.

In some cases, the wearable device 104 may measure a first signal (e.g., an input signal). The first signal may include one or more physiological phenomena and a noise component. The first signal may undergo a DWT, where the input signal may be decomposed into various sets of coefficients, where each set of coefficients represents a specific frequency range (e.g., first set of coefficients associated with first frequency range, second set of coefficients associated with second frequency range, etc.). The respective “sets of coefficients” may additionally or alternatively be referred to as “components.” The DWT may filter out noise by eliminating higher frequencies (or otherwise filtering out frequencies that fall outside of some threshold frequency range), as well as through a noise thresholding mechanism. A device (e.g., a wearable device 104, a device associated with the wearable device 104 such as a server 110 or user device 106) may accumulate one or more of these anomalies and construct a noise reference, which may often be encountered during sporadic movements (e.g., hand gesture) by the user 102.

In additional or alternative cases, a wearable device 104 may measure a first signal including one or more physiological phenomena and a noise component. A device (e.g., the wearable device 104, user device 106, server 110, etc.) may establish a noise reference by utilizing an accelerometer to gain a deeper understanding of photoplethysmography (PPG) motion. In this scenario, the device may obtain different signals (e.g., from distinct light emitting elements) that then undergo an ICA. Through this analysis, various ICA components, such as independent PPG sources like movements, steps, and heart rate, may be extracted from the initial signals. In some instances, the signals may serve as inputs, and the ICA components may become the outputs. Once the device identifies which ICA components correlate with the accelerometer measurements, the device may construct a noise reference. Additionally, or alternatively, the device may compare the different physiological measurements to ascertain a noise component based on establishing a noise reference and determining whether to delete one or more signals. Thus, the device may effectively filter the noise from the prior measurements by eliminating one or more noise components, which may result in a cleaner signal that may be used to more accurately evaluate physiological characteristics/phenomena of the user.

It should be appreciated by a person skilled in the art that one or more aspects of the disclosure may be implemented in a system 100 to additionally or alternatively solve other problems than those described above. Furthermore, aspects of the disclosure may provide technical improvements to “conventional” systems or processes as described herein. However, the description and appended drawings only include example technical improvements resulting from implementing aspects of the disclosure, and accordingly do not represent all of the technical improvements provided within the scope of the claims.

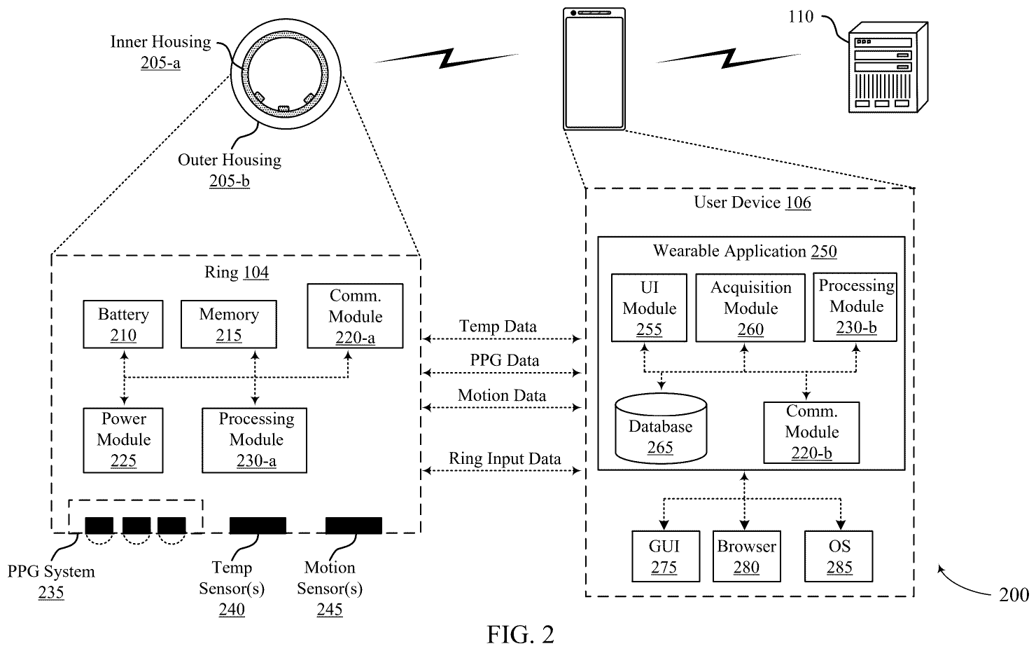

FIG. 2 illustrates an example of a system 200 that supports techniques for noise reduction in accordance with aspects of the present disclosure. The system 200 may implement, or be implemented by, system 100. In particular, system 200 illustrates an example of a ring 104 (e.g., wearable device 104), a user device 106, and a server 110, as described with reference to FIG. 1.

In some aspects, the ring 104 may be configured to be worn around a user's finger, and may determine one or more user physiological parameters when worn around the user's finger. Example measurements and determinations may include, but are not limited to, user skin temperature, pulse waveforms, respiratory rate, heart rate, HRV, blood oxygen levels (SpO2), blood sugar levels (e.g., glucose metrics), and the like.

The system 200 further includes a user device 106 (e.g., a smartphone) in communication with the ring 104. For example, the ring 104 may be in wireless and/or wired communication with the user device 106. In some implementations, the ring 104 may send measured and processed data (e.g., temperature data, photoplethysmogram (PPG) data, motion/accelerometer data, ring input data, and the like) to the user device 106. The user device 106 may also send data to the ring 104, such as ring 104 firmware/configuration updates. The user device 106 may process data. In some implementations, the user device 106 may transmit data to the server 110 for processing and/or storage.

The ring 104 may include a housing 205 that may include an inner housing 205-a and an outer housing 205-b. In some aspects, the housing 205 of the ring 104 may store or otherwise include various components of the ring including, but not limited to, device electronics, a power source (e.g., battery 210, and/or capacitor), one or more substrates (e.g., printable circuit boards) that interconnect the device electronics and/or power source, and the like. The device electronics may include device modules (e.g., hardware/software), such as: a processing module 230-a, a memory 215, a communication module 220-a, a power module 225, and the like. The device electronics may also include one or more sensors. Example sensors may include one or more temperature sensors 240, a PPG sensor assembly (e.g., PPG system 235), and one or more motion sensors 245.

The sensors may include associated modules (not illustrated) configured to communicate with the respective components/modules of the ring 104, and generate signals associated with the respective sensors. In some aspects, each of the components/modules of the ring 104 may be communicatively coupled to one another via wired or wireless connections. Moreover, the ring 104 may include additional and/or alternative sensors or other components that are configured to collect physiological data from the user, including light sensors (e.g., LEDs), oximeters, and the like.

The ring 104 shown and described with reference to FIG. 2 is provided solely for illustrative purposes. As such, the ring 104 may include additional or alternative components as those illustrated in FIG. 2. Other rings 104 that provide functionality described herein may be fabricated. For example, rings 104 with fewer components (e.g., sensors) may be fabricated. In a specific example, a ring 104 with a single temperature sensor 240 (or other sensor), a power source, and device electronics configured to read the single temperature sensor 240 (or other sensor) may be fabricated. In another specific example, a temperature sensor 240 (or other sensor) may be attached to a user's finger (e.g., using adhesives, wraps, clamps, spring loaded clamps, etc.). In this case, the sensor may be wired to another computing device, such as a wrist worn computing device that reads the temperature sensor 240 (or other sensor). In other examples, a ring 104 that includes additional sensors and processing functionality may be fabricated.

The housing 205 may include one or more housing 205 components. The housing 205 may include an outer housing 205-b component (e.g., a shell) and an inner housing 205-a component (e.g., a molding). The housing 205 may include additional components (e.g., additional layers) not explicitly illustrated in FIG. 2. For example, in some implementations, the ring 104 may include one or more insulating layers that electrically insulate the device electronics and other conductive materials (e.g., electrical traces) from the outer housing 205-b (e.g., a metal outer housing 205-b). The housing 205 may provide structural support for the device electronics, battery 210, substrate(s), and other components. For example, the housing 205 may protect the device electronics, battery 210, and substrate(s) from mechanical forces, such as pressure and impacts. The housing 205 may also protect the device electronics, battery 210, and substrate(s) from water and/or other chemicals.

The outer housing 205-b may be fabricated from one or more materials. In some implementations, the outer housing 205-b may include a metal, such as titanium, that may provide strength and abrasion resistance at a relatively light weight. The outer housing 205-b may also be fabricated from other materials, such polymers. In some implementations, the outer housing 205-b may be protective as well as decorative.

The inner housing 205-a may be configured to interface with the user's finger. The inner housing 205-a may be formed from a polymer (e.g., a medical grade polymer) or other material. In some implementations, the inner housing 205-a may be transparent. For example, the inner housing 205-a may be transparent to light emitted by the PPG light emitting diodes (LEDs). In some implementations, the inner housing 205-a component may be molded onto the outer housing 205-b. For example, the inner housing 205-a may include a polymer that is molded (e.g., injection molded) to fit into an outer housing 205-b metallic shell.

The ring 104 may include one or more substrates (not illustrated). The device electronics and battery 210 may be included on the one or more substrates. For example, the device electronics and battery 210 may be mounted on one or more substrates. Example substrates may include one or more printed circuit boards (PCBs), such as flexible PCB (e.g., polyimide). In some implementations, the electronics/battery 210 may include surface mounted devices (e.g., surface-mount technology (SMT) devices) on a flexible PCB. In some implementations, the one or more substrates (e.g., one or more flexible PCBs) may include electrical traces that provide electrical communication between device electronics. The electrical traces may also connect the battery 210 to the device electronics.

The device electronics, battery 210, and substrates may be arranged in the ring 104 in a variety of ways. In some implementations, one substrate that includes device electronics may be mounted along the bottom of the ring 104 (e.g., the bottom half), such that the sensors (e.g., PPG system 235, temperature sensors 240, motion sensors 245, and other sensors) interface with the underside of the user's finger. In these implementations, the battery 210 may be included along the top portion of the ring 104 (e.g., on another substrate).

The various components/modules of the ring 104 represent functionality (e.g., circuits and other components) that may be included in the ring 104. Modules may include any discrete and/or integrated electronic circuit components that implement analog and/or digital circuits capable of producing the functions attributed to the modules herein. For example, the modules may include analog circuits (e.g., amplification circuits, filtering circuits, analog/digital conversion circuits, and/or other signal conditioning circuits). The modules may also include digital circuits (e.g., combinational or sequential logic circuits, memory circuits etc.).

The memory 215 (memory module) of the ring 104 may include any volatile, non-volatile, magnetic, or electrical media, such as a random access memory (RAM), read-only memory (ROM), non-volatile RAM (NVRAM), electrically-erasable programmable ROM (EEPROM), flash memory, or any other memory device. The memory 215 may store any of the data described herein. For example, the memory 215 may be configured to store data (e.g., motion data, temperature data, PPG data) collected by the respective sensors and PPG system 235. Furthermore, memory 215 may include instructions that, when executed by one or more processing circuits, cause the modules to perform various functions attributed to the modules herein. The device electronics of the ring 104 described herein are only example device electronics. As such, the types of electronic components used to implement the device electronics may vary based on design considerations.

The functions attributed to the modules of the ring 104 described herein may be embodied as one or more processors, hardware, firmware, software, or any combination thereof. Depiction of different features as modules is intended to highlight different functional aspects and does not necessarily imply that such modules must be realized by separate hardware/software components. Rather, functionality associated with one or more modules may be performed by separate hardware/software components or integrated within common hardware/software components.

The processing module 230-a of the ring 104 may include one or more processors (e.g., processing units), microcontrollers, digital signal processors, systems on a chip (SOCs), and/or other processing devices. The processing module 230-a communicates with the modules included in the ring 104. For example, the processing module 230-a may transmit/receive data to/from the modules and other components of the ring 104, such as the sensors. As described herein, the modules may be implemented by various circuit components. Accordingly, the modules may also be referred to as circuits (e.g., a communication circuit and power circuit).

The processing module 230-a may communicate with the memory 215. The memory 215 may include computer-readable instructions that, when executed by the processing module 230-a, cause the processing module 230-a to perform the various functions attributed to the processing module 230-a herein. In some implementations, the processing module 230-a (e.g., a microcontroller) may include additional features associated with other modules, such as communication functionality provided by the communication module 220-a (e.g., an integrated Bluetooth Low Energy transceiver) and/or additional onboard memory 215.

The communication module 220-a may include circuits that provide wireless and/or wired communication with the user device 106 (e.g., communication module 220-b of the user device 106). In some implementations, the communication modules 220-a, 220-b may include wireless communication circuits, such as Bluetooth circuits and/or Wi-Fi circuits. In some implementations, the communication modules 220-a, 220-b can include wired communication circuits, such as Universal Serial Bus (USB) communication circuits. Using the communication module 220-a, the ring 104 and the user device 106 may be configured to communicate with each other. The processing module 230-a of the ring may be configured to transmit/receive data to/from the user device 106 via the communication module 220-a. Example data may include, but is not limited to, motion data, temperature data, pulse waveforms, heart rate data, HRV data, PPG data, and status updates (e.g., charging status, battery charge level, and/or ring 104 configuration settings). The processing module 230-a of the ring may also be configured to receive updates (e.g., software/firmware updates) and data from the user device 106.

The ring 104 may include a battery 210 (e.g., a rechargeable battery 210). An example battery 210 may include a Lithium-Ion or Lithium-Polymer type battery 210, although a variety of battery 210 options are possible. The battery 210 may be wirelessly charged. In some implementations, the ring 104 may include a power source other than the battery 210, such as a capacitor. The power source (e.g., battery 210 or capacitor) may have a curved geometry that matches the curve of the ring 104. In some aspects, a charger or other power source may include additional sensors that may be used to collect data in addition to, or that supplements, data collected by the ring 104 itself. Moreover, a charger or other power source for the ring 104 may function as a user device 106, in which case the charger or other power source for the ring 104 may be configured to receive data from the ring 104, store and/or process data received from the ring 104, and communicate data between the ring 104 and the servers 110.

In some aspects, the ring 104 includes a power module 225 that may control charging of the battery 210. For example, the power module 225 may interface with an external wireless charger that charges the battery 210 when interfaced with the ring 104. The charger may include a datum structure that mates with a ring 104 datum structure to create a specified orientation with the ring 104 during charging. The power module 225 may also regulate voltage(s) of the device electronics, regulate power output to the device electronics, and monitor the state of charge of the battery 210. In some implementations, the battery 210 may include a protection circuit module (PCM) that protects the battery 210 from high current discharge, over voltage during charging, and under voltage during discharge. The power module 225 may also include electro-static discharge (ESD) protection.

The one or more temperature sensors 240 may be electrically coupled to the processing module 230-a. The temperature sensor 240 may be configured to generate a temperature signal (e.g., temperature data) that indicates a temperature read or sensed by the temperature sensor 240. The processing module 230-a may determine a temperature of the user in the location of the temperature sensor 240. For example, in the ring 104, temperature data generated by the temperature sensor 240 may indicate a temperature of a user at the user's finger (e.g., skin temperature). In some implementations, the temperature sensor 240 may contact the user's skin. In other implementations, a portion of the housing 205 (e.g., the inner housing 205-a) may form a barrier (e.g., a thin, thermally conductive barrier) between the temperature sensor 240 and the user's skin. In some implementations, portions of the ring 104 configured to contact the user's finger may have thermally conductive portions and thermally insulative portions. The thermally conductive portions may conduct heat from the user's finger to the temperature sensors 240. The thermally insulative portions may insulate portions of the ring 104 (e.g., the temperature sensor 240) from ambient temperature.

In some implementations, the temperature sensor 240 may generate a digital signal (e.g., temperature data) that the processing module 230-a may use to determine the temperature. As another example, in cases where the temperature sensor 240 includes a passive sensor, the processing module 230-a (or a temperature sensor 240 module) may measure a current/voltage generated by the temperature sensor 240 and determine the temperature based on the measured current/voltage. Example temperature sensors 240 may include a thermistor, such as a negative temperature coefficient (NTC) thermistor, or other types of sensors including resistors, transistors, diodes, and/or other electrical/electronic components.

The processing module 230-a may sample the user's temperature over time. For example, the processing module 230-a may sample the user's temperature according to a sampling rate. An example sampling rate may include one sample per second, although the processing module 230-a may be configured to sample the temperature signal at other sampling rates that are higher or lower than one sample per second. In some implementations, the processing module 230-a may sample the user's temperature continuously throughout the day and night. Sampling at a sufficient rate (e.g., one sample per second) throughout the day may provide sufficient temperature data for analysis described herein.

The processing module 230-a may store the sampled temperature data in memory 215. In some implementations, the processing module 230-a may process the sampled temperature data. For example, the processing module 230-a may determine average temperature values over a period of time. In one example, the processing module 230-a may determine an average temperature value each minute by summing all temperature values collected over the minute and dividing by the number of samples over the minute. In a specific example where the temperature is sampled at one sample per second, the average temperature may be a sum of all sampled temperatures for one minute divided by sixty seconds. The memory 215 may store the average temperature values over time. In some implementations, the memory 215 may store average temperatures (e.g., one per minute) instead of sampled temperatures in order to conserve memory 215.

The sampling rate, which may be stored in memory 215, may be configurable. In some implementations, the sampling rate may be the same throughout the day and night. In other implementations, the sampling rate may be changed throughout the day/night. In some implementations, the ring 104 may filter/reject temperature readings, such as large spikes in temperature that are not indicative of physiological changes (e.g., a temperature spike from a hot shower). In some implementations, the ring 104 may filter/reject temperature readings that may not be reliable due to other factors, such as excessive motion during exercise (e.g., as indicated by a motion sensor 245).

The ring 104 (e.g., communication module) may transmit the sampled and/or average temperature data to the user device 106 for storage and/or further processing. The user device 106 may transfer the sampled and/or average temperature data to the server 110 for storage and/or further processing.

Although the ring 104 is illustrated as including a single temperature sensor 240, the ring 104 may include multiple temperature sensors 240 in one or more locations, such as arranged along the inner housing 205-a near the user's finger. In some implementations, the temperature sensors 240 may be stand-alone temperature sensors 240. Additionally, or alternatively, one or more temperature sensors 240 may be included with other components (e.g., packaged with other components), such as with the accelerometer and/or processor.

The processing module 230-a may acquire and process data from multiple temperature sensors 240 in a similar manner described with respect to a single temperature sensor 240. For example, the processing module 230 may individually sample, average, and store temperature data from each of the multiple temperature sensors 240. In other examples, the processing module 230-a may sample the sensors at different rates and average/store different values for the different sensors. In some implementations, the processing module 230-a may be configured to determine a single temperature based on the average of two or more temperatures determined by two or more temperature sensors 240 in different locations on the finger.

The temperature sensors 240 on the ring 104 may acquire distal temperatures at the user's finger (e.g., any finger). For example, one or more temperature sensors 240 on the ring 104 may acquire a user's temperature from the underside of a finger or at a different location on the finger. In some implementations, the ring 104 may continuously acquire distal temperature (e.g., at a sampling rate). Although distal temperature measured by a ring 104 at the finger is described herein, other devices may measure temperature at the same/different locations. In some cases, the distal temperature measured at a user's finger may differ from the temperature measured at a user's wrist or other external body location. Additionally, the distal temperature measured at a user's finger (e.g., a “shell” temperature) may differ from the user's core temperature. As such, the ring 104 may provide a useful temperature signal that may not be acquired at other internal/external locations of the body. In some cases, continuous temperature measurement at the finger may capture temperature fluctuations (e.g., small or large fluctuations) that may not be evident in core temperature. For example, continuous temperature measurement at the finger may capture minute-to-minute or hour-to-hour temperature fluctuations that provide additional insight that may not be provided by other temperature measurements elsewhere in the body.

The ring 104 may include a PPG system 235. The PPG system 235 may include one or more optical transmitters that transmit light. The PPG system 235 may also include one or more optical receivers that receive light transmitted by the one or more optical transmitters. An optical receiver may generate a signal (hereinafter “PPG” signal) that indicates an amount of light received by the optical receiver. The optical transmitters may illuminate a region of the user's finger. The PPG signal generated by the PPG system 235 may indicate the perfusion of blood in the illuminated region. For example, the PPG signal may indicate blood volume changes in the illuminated region caused by a user's pulse pressure. The processing module 230-a may sample the PPG signal and determine a user's pulse waveform based on the PPG signal. The processing module 230-a may determine a variety of physiological parameters based on the user's pulse waveform, such as a user's respiratory rate, heart rate, HRV, oxygen saturation, and other circulatory parameters.

In some implementations, the PPG system 235 may be configured as a reflective PPG system 235 where the optical receiver(s) receive transmitted light that is reflected through the region of the user's finger. In some implementations, the PPG system 235 may be configured as a transmissive PPG system 235 where the optical transmitter(s) and optical receiver(s) are arranged opposite to one another, such that light is transmitted directly through a portion of the user's finger to the optical receiver(s).

The number and ratio of transmitters and receivers included in the PPG system 235 may vary. Example optical transmitters may include light-emitting diodes (LEDs). The optical transmitters may transmit light in the infrared spectrum and/or other spectrums. Example optical receivers may include, but are not limited to, photosensors, phototransistors, and photodiodes. The optical receivers may be configured to generate PPG signals in response to the wavelengths received from the optical transmitters. The location of the transmitters and receivers may vary. Additionally, a single device may include reflective and/or transmissive PPG systems 235.

The PPG system 235 illustrated in FIG. 2 may include a reflective PPG system 235 in some implementations. In these implementations, the PPG system 235 may include a centrally located optical receiver (e.g., at the bottom of the ring 104) and two optical transmitters located on each side of the optical receiver. In this implementation, the PPG system 235 (e.g., optical receiver) may generate the PPG signal based on light received from one or both of the optical transmitters. In other implementations, other placements, combinations, and/or configurations of one or more optical transmitters and/or optical receivers are contemplated.

The processing module 230-a may control one or both of the optical transmitters to transmit light while sampling the PPG signal generated by the optical receiver. In some implementations, the processing module 230-a may cause the optical transmitter with the stronger received signal to transmit light while sampling the PPG signal generated by the optical receiver. For example, the selected optical transmitter may continuously emit light while the PPG signal is sampled at a sampling rate (e.g., 250 Hz).

Sampling the PPG signal generated by the PPG system 235 may result in a pulse waveform that may be referred to as a “PPG.” The pulse waveform may indicate blood pressure vs time for multiple cardiac cycles. The pulse waveform may include peaks that indicate cardiac cycles. Additionally, the pulse waveform may include respiratory induced variations that may be used to determine respiration rate. The processing module 230-a may store the pulse waveform in memory 215 in some implementations. The processing module 230-a may process the pulse waveform as it is generated and/or from memory 215 to determine user physiological parameters described herein.

The processing module 230-a may determine the user's heart rate based on the pulse waveform. For example, the processing module 230-a may determine heart rate (e.g., in beats per minute) based on the time between peaks in the pulse waveform. The time between peaks may be referred to as an interbeat interval (IBI). The processing module 230-a may store the determined heart rate values and IBI values in memory 215.

The processing module 230-a may determine HRV over time. For example, the processing module 230-a may determine HRV based on the variation in the IBIs. The processing module 230-a may store the HRV values over time in the memory 215. Moreover, the processing module 230-a may determine the user's respiratory rate over time. For example, the processing module 230-a may determine respiratory rate based on frequency modulation, amplitude modulation, or baseline modulation of the user's IBI values over a period of time. Respiratory rate may be calculated in breaths per minute or as another breathing rate (e.g., breaths per 30 seconds). The processing module 230-a may store user respiratory rate values over time in the memory 215.

The ring 104 may include one or more motion sensors 245, such as one or more accelerometers (e.g., 6-D accelerometers) and/or one or more gyroscopes (gyros). The motion sensors 245 may generate motion signals that indicate motion of the sensors. For example, the ring 104 may include one or more accelerometers that generate acceleration signals that indicate acceleration of the accelerometers. As another example, the ring 104 may include one or more gyro sensors that generate gyro signals that indicate angular motion (e.g., angular velocity) and/or changes in orientation. The motion sensors 245 may be included in one or more sensor packages. An example accelerometer/gyro sensor is a Bosch BM1160 inertial micro electro-mechanical system (MEMS) sensor that may measure angular rates and accelerations in three perpendicular axes.

The processing module 230-a may sample the motion signals at a sampling rate (e.g., 50 Hz) and determine the motion of the ring 104 based on the sampled motion signals. For example, the processing module 230-a may sample acceleration signals to determine acceleration of the ring 104. As another example, the processing module 230-a may sample a gyro signal to determine angular motion. In some implementations, the processing module 230-a may store motion data in memory 215. Motion data may include sampled motion data as well as motion data that is calculated based on the sampled motion signals (e.g., acceleration and angular values).

The ring 104 may store a variety of data described herein. For example, the ring 104 may store temperature data, such as raw sampled temperature data and calculated temperature data (e.g., average temperatures). As another example, the ring 104 may store PPG signal data, such as pulse waveforms and data calculated based on the pulse waveforms (e.g., heart rate values, IBI values, HRV values, and respiratory rate values). The ring 104 may also store motion data, such as sampled motion data that indicates linear and angular motion.

The ring 104, or other computing device, may calculate and store additional values based on the sampled/calculated physiological data. For example, the processing module 230 may calculate and store various metrics, such as sleep metrics (e.g., a Sleep Score), activity metrics, and readiness metrics. In some implementations, additional values/metrics may be referred to as “derived values.” The ring 104, or other computing/wearable device, may calculate a variety of values/metrics with respect to motion. Example derived values for motion data may include, but are not limited to, motion count values, regularity values, intensity values, metabolic equivalence of task values (METs), and orientation values. Motion counts, regularity values, intensity values, and METs may indicate an amount of user motion (e.g., velocity/acceleration) over time. Orientation values may indicate how the ring 104 is oriented on the user's finger and if the ring 104 is worn on the left hand or right hand.

In some implementations, motion counts and regularity values may be determined by counting a number of acceleration peaks within one or more periods of time (e.g., one or more 30 second to 1 minute periods). Intensity values may indicate a number of movements and the associated intensity (e.g., acceleration values) of the movements. The intensity values may be categorized as low, medium, and high, depending on associated threshold acceleration values. METs may be determined based on the intensity of movements during a period of time (e.g., 30 seconds), the regularity/irregularity of the movements, and the number of movements associated with the different intensities.

In some implementations, the processing module 230-a may compress the data stored in memory 215. For example, the processing module 230-a may delete sampled data after making calculations based on the sampled data. As another example, the processing module 230-a may average data over longer periods of time in order to reduce the number of stored values. In a specific example, if average temperatures for a user over one minute are stored in memory 215, the processing module 230-a may calculate average temperatures over a five minute time period for storage, and then subsequently erase the one minute average temperature data. The processing module 230-a may compress data based on a variety of factors, such as the total amount of used/available memory 215 and/or an elapsed time since the ring 104 last transmitted the data to the user device 106.

Although a user's physiological parameters may be measured by sensors included on a ring 104, other devices may measure a user's physiological parameters. For example, although a user's temperature may be measured by a temperature sensor 240 included in a ring 104, other devices may measure a user's temperature. In some examples, other wearable devices (e.g., wrist devices) may include sensors that measure user physiological parameters. Additionally, medical devices, such as external medical devices (e.g., wearable medical devices) and/or implantable medical devices, may measure a user's physiological parameters. One or more sensors on any type of computing device may be used to implement the techniques described herein.

The physiological measurements may be taken continuously throughout the day and/or night. In some implementations, the physiological measurements may be taken during portions of the day and/or portions of the night. In some implementations, the physiological measurements may be taken in response to determining that the user is in a specific state, such as an active state, resting state, and/or a sleeping state. For example, the ring 104 can make physiological measurements in a resting/sleep state in order to acquire cleaner physiological signals. In one example, the ring 104 or other device/system may detect when a user is resting and/or sleeping and acquire physiological parameters (e.g., temperature) for that detected state. The devices/systems may use the resting/sleep physiological data and/or other data when the user is in other states in order to implement the techniques of the present disclosure.

In some implementations, as described previously herein, the ring 104 may be configured to collect, store, and/or process data, and may transfer any of the data described herein to the user device 106 for storage and/or processing. In some aspects, the user device 106 includes a wearable application 250, an operating system (OS), a web browser application (e.g., web browser 280), one or more additional applications, and a GUI 275. The user device 106 may further include other modules and components, including sensors, audio devices, haptic feedback devices, and the like. The wearable application 250 may include an example of an application (e.g., “app”) that may be installed on the user device 106. The wearable application 250 may be configured to acquire data from the ring 104, store the acquired data, and process the acquired data as described herein. For example, the wearable application 250 may include a user interface (UI) module 255, an acquisition module 260, a processing module 230-b, a communication module 220-b, and a storage module (e.g., database 265) configured to store application data.

In some cases, the wearable device 104 and the user device 106 may be included within (or make up) the same device. For example, in some cases, the wearable device 104 may be configured to execute the wearable application 250, and may be configured to display data via the GUI 275.

The various data processing operations described herein may be performed by the ring 104, the user device 106, the servers 110, or any combination thereof. For example, in some cases, data collected by the ring 104 may be pre-processed and transmitted to the user device 106. In this example, the user device 106 may perform some data processing operations on the received data, may transmit the data to the servers 110 for data processing, or both. For instance, in some cases, the user device 106 may perform processing operations that require relatively low processing power and/or operations that require a relatively low latency, whereas the user device 106 may transmit the data to the servers 110 for processing operations that require relatively high processing power and/or operations that may allow relatively higher latency.

In some aspects, the ring 104, user device 106, and server 110 of the system 200 may be configured to evaluate sleep patterns for a user. In particular, the respective components of the system 200 may be used to collect data from a user via the ring 104, and generate one or more scores (e.g., Sleep Score, Readiness Score) for the user based on the collected data. For example, as noted previously herein, the ring 104 of the system 200 may be worn by a user to collect data from the user, including temperature, heart rate, HRV, and the like. Data collected by the ring 104 may be used to determine when the user is asleep in order to evaluate the user's sleep for a given “sleep day.” In some aspects, scores may be calculated for the user for each respective sleep day, such that a first sleep day is associated with a first set of scores, and a second sleep day is associated with a second set of scores. Scores may be calculated for each respective sleep day based on data collected by the ring 104 during the respective sleep day. Scores may include, but are not limited to, Sleep Scores, Readiness Scores, and the like.

In some cases, “sleep days” may align with the traditional calendar days, such that a given sleep day runs from midnight to midnight of the respective calendar day. In other cases, sleep days may be offset relative to calendar days. For example, sleep days may run from 6:00 pm (18:00) of a calendar day until 6:00 pm (18:00) of the subsequent calendar day. In this example, 6:00 pm may serve as a “cut-off time,” where data collected from the user before 6:00 pm is counted for the current sleep day, and data collected from the user after 6:00 pm is counted for the subsequent sleep day. Due to the fact that most individuals sleep the most at night, offsetting sleep days relative to calendar days may enable the system 200 to evaluate sleep patterns for users in such a manner that is consistent with their sleep schedules. In some cases, users may be able to selectively adjust (e.g., via the GUI) a timing of sleep days relative to calendar days so that the sleep days are aligned with the duration of time that the respective users typically sleep.

In some implementations, each overall score for a user for each respective day (e.g., Sleep Score, Readiness Score) may be determined/calculated based on one or more “contributors,” “factors,” or “contributing factors.” For example, a user's overall Sleep Score may be calculated based on a set of contributors, including: total sleep, efficiency, restfulness, REM sleep, deep sleep, latency, timing, or any combination thereof. The Sleep Score may include any quantity of contributors. The “total sleep” contributor may refer to the sum of all sleep periods of the sleep day. The “efficiency” contributor may reflect the percentage of time spent asleep compared to time spent awake while in bed, and may be calculated using the efficiency average of long sleep periods (e.g., primary sleep period) of the sleep day, weighted by a duration of each sleep period. The “restfulness” contributor may indicate how restful the user's sleep is, and may be calculated using the average of all sleep periods of the sleep day, weighted by a duration of each period. The restfulness contributor may be based on a “wake up count” (e.g., sum of all the wake-ups (when user wakes up) detected during different sleep periods), excessive movement, and a “got up count” (e.g., sum of all the got-ups (when user gets out of bed) detected during the different sleep periods).

The “REM sleep” contributor may refer to a sum total of REM sleep durations across all sleep periods of the sleep day including REM sleep. Similarly, the “deep sleep” contributor may refer to a sum total of deep sleep durations across all sleep periods of the sleep day including deep sleep. The “latency” contributor may signify how long (e.g., average, median, longest) the user takes to go to sleep, and may be calculated using the average of long sleep periods throughout the sleep day, weighted by a duration of each period and the number of such periods (e.g., consolidation of a given sleep stage or sleep stages may be its own contributor or weight other contributors). Lastly, the “timing” contributor may refer to a relative timing of sleep periods within the sleep day and/or calendar day, and may be calculated using the average of all sleep periods of the sleep day, weighted by a duration of each period.

By way of another example, a user's overall Readiness Score may be calculated based on a set of contributors, including: sleep, sleep balance, heart rate, HRV balance, recovery index, temperature, activity, activity balance, or any combination thereof. The Readiness Score may include any quantity of contributors. The “sleep” contributor may refer to the combined Sleep Score of all sleep periods within the sleep day. The “sleep balance” contributor may refer to a cumulative duration of all sleep periods within the sleep day. In particular, sleep balance may indicate to a user whether the sleep that the user has been getting over some duration of time (e.g., the past two weeks) is in balance with the user's needs. Typically, adults need 7-9 hours of sleep a night to stay healthy, alert, and to perform at their best both mentally and physically. However, it is normal to have an occasional night of bad sleep, so the sleep balance contributor takes into account long-term sleep patterns to determine whether each user's sleep needs are being met. The “resting heart rate” contributor may indicate a lowest heart rate from the longest sleep period of the sleep day (e.g., primary sleep period) and/or the lowest heart rate from naps occurring after the primary sleep period.

Continuing with reference to the “contributors” (e.g., factors, contributing factors) of the Readiness Score, the “HRV balance” contributor may indicate a highest HRV average from the primary sleep period and the naps happening after the primary sleep period. The HRV balance contributor may help users keep track of their recovery status by comparing their HRV trend over a first time period (e.g., two weeks) to an average HRV over some second, longer time period (e.g., three months). The “recovery index” contributor may be calculated based on the longest sleep period. Recovery index measures how long it takes for a user's resting heart rate to stabilize during the night. A sign of a very good recovery is that the user's resting heart rate stabilizes during the first half of the night, at least six hours before the user wakes up, leaving the body time to recover for the next day. The “body temperature” contributor may be calculated based on the longest sleep period (e.g., primary sleep period) or based on a nap happening after the longest sleep period if the user's highest temperature during the nap is at least 0.5° C. higher than the highest temperature during the longest period. In some aspects, the ring may measure a user's body temperature while the user is asleep, and the system 200 may display the user's average temperature relative to the user's baseline temperature. If a user's body temperature is outside of their normal range (e.g., clearly above or below 0.0), the body temperature contributor may be highlighted (e.g., go to a “Pay attention” state) or otherwise generate an alert for the user.

In some aspects, the system 200 may support techniques for employing computation and mathematical techniques to filter noise from signals/measurements acquired via a wearable device 104. For example, physiological data, motion/mobility measurements, or both, acquired by a wearable device 104 may be subject to noise from the variability in the different sensors (e.g., sensors of the PPG system 235). In this regard, respective devices of the system 200 (e.g., wearable device 104, user device 106, servers 110, etc.) may perform a noise filtering procedure that employs one or more mathematical (e.g., computational) techniques or algorithms aimed at improving the quality of the acquired signals.

In some cases, the wearable device 104 may measure a first signal (e.g., an input signal). The first signal may include one or more physiological phenomena and a noise component. The first signal may undergo a DWT, where the input signal may be decomposed into various sets of coefficients, where each set of coefficients represents a specific frequency range. The respective “sets of coefficients” may additionally or alternatively be referred to as “components.” The DWT may filter out noise by eliminating higher frequencies, as well as through a noise thresholding mechanism. The system 200 (e.g., wearable device 104, user device 106, servers 110, etc.) may accumulate one or more of these anomalies and construct a noise reference, which may often be encountered during sporadic movements (e.g., hand gesture) by the user 102.

In additional or alternative implementations, a wearable device 104 may measure a first signal including one or more physiological phenomena and a noise component. The system 200 (e.g., wearable device 104, user device 106, servers 110, etc.) may establish a noise reference by utilizing an accelerometer to gain a deeper understanding of PPG motion. In this scenario, the system 200 may obtain different signals (e.g., from distinct light emitting elements) that may undergo an ICA. Through this analysis, various ICA components, such as independent PPG sources like movements, steps, and heart rate, may be extracted from the initial signals. In some instances, the signals may serve as inputs, and the ICA components may become the outputs. Once the system 200 identifies which ICA components correlate with the accelerometer measurements, the system 200 may construct a noise reference. Additionally, or alternatively, the system 200 may compare the different physiological measurements to ascertain a noise component based on establishing a noise reference and determining whether to delete one or more signals. Thus, the system 200 may effectively filter the noise from the prior measurements by eliminating one or more noise components, which may result in a cleaner signal that may be used to more accurately/reliably evaluate physiological characteristics/phenomena of the user 102.

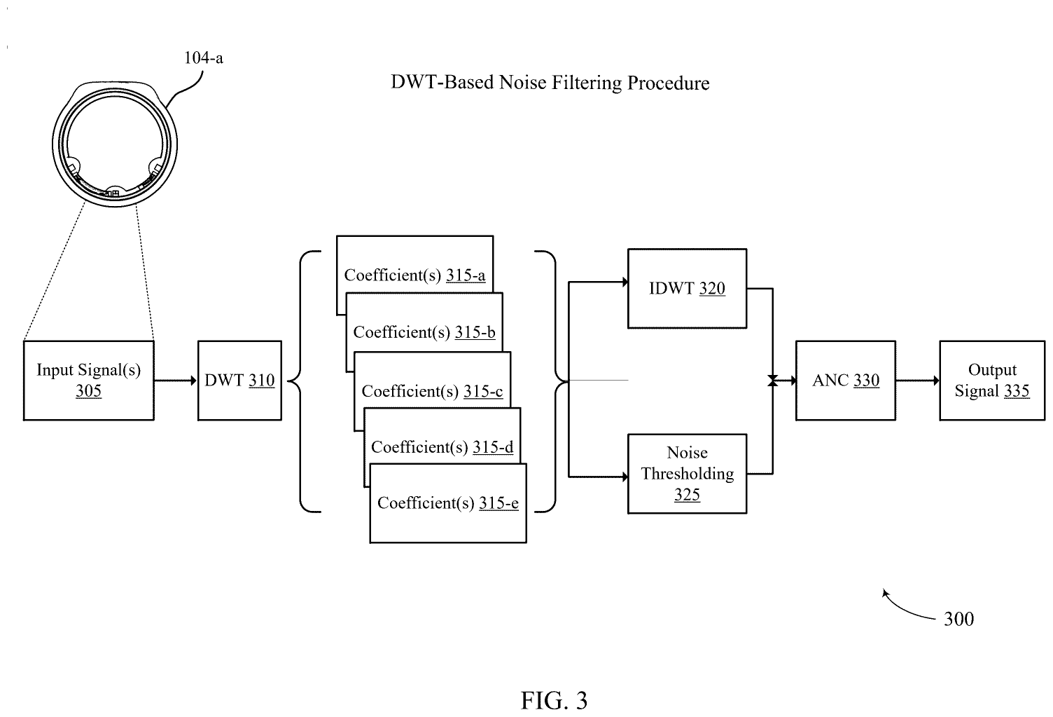

FIG. 3 shows an example of a noise filtering procedure 300 that supports techniques for noise reduction in accordance with aspects of the present disclosure. The noise filtering procedure 300 (e.g., filtering procedure) may implement, or be implemented by, aspects of the system 100, the system 200, or both. The noise filtering procedure 300 shown and described in FIG. 3 may be performed at a wearable device 104-a, a user device 106, one or more servers 110, or any combination thereof, as described with reference to FIG. 1 and FIG. 2. The wearable device 104-a may be any example of a wearable ring device 104, a watch or other wrist-worn wearable device (e.g., bracelet), a necklace, and the like.

The noise filtering procedure 300 illustrated in FIG. 3 illustrates an example of a DWT-based noise filtering procedure that may be used to reduce or eliminate noise from signals/measurements performed via a wearable device 104-a. In some cases, the noise filtering procedure 300 may effectively eliminate one or more noise components (e.g., one or more sets of coefficients) from signals acquired by the wearable device 104-a, and may result in a clean signal that may be used to more accurately and reliably perform physiological measurements of the user 102 (e.g., heart rate measurements, HRV measurements, blood oxygen measurements, blood pressure measurements, etc.).

For example, a wearable device 104-a may emit light from a set of light emitting elements (e.g., one or more LEDs), and may receive the light reflected/refracted from a tissue of a user 102 using one or more photodetectors. The photodetectors may generate one or more first signals (e.g., input signal(s) 305) that is based on the light received via the photodetectors. The input signal 305 may include a representation of one or more physiological phenomena of the user 102 (e.g., heart rate measurements, blood oxygen levels measurements). The input signal 305 may further include a noise component associated with the set of light emitting elements, the photodetectors, or both. The input signal 305 (e.g., signals acquired by the wearable device 104-a) may be measured over a specified time window, where the noise component of the input signal 305 may be associated with or attributable to movement (e.g., user motion) during the time interval. For example, movement of the user's hand, finger, etc., as well as movement of the wearable device 104-a relative to the user's tissue (e.g., the ring rotating around the user's finger) may result in noise/motion artifacts within the acquired input signal 305.

In some cases, the noise filtering procedure 300 may involve processing the input signal(s) 305. For example, the noise filtering procedure 300 may process the input signal(s) 305 to filter (e.g., remove or eliminate) one or more characteristics from the input signal(s) 305, including the noise component. The noise filtering procedure 300 may include a DWT 310 (e.g., DWT-based noise filtering procedure 300). For example, the noise filtering procedure 300 may decompose the input signal 305 into one or more sets of coefficients 315 (e.g., a first set of coefficients 315-a, a second set of coefficients 315-b, a third set of coefficients 315-c, a fourth set of coefficients 315-d, and a fifth set of coefficients 315-e). In some instances, each set of coefficients 315 may be associated with (e.g., represent) a respective frequency range within the input signal(s) 305. The respective sets of coefficients 315 may additionally or alternatively be referred to as “components.”

For instance, the first set of coefficients 315-a (e.g., first component) may represent a first frequency range, the second set of coefficients 315-b (e.g., second component) may represent a second frequency range, the third set of coefficients 315-c (e.g., third component) may represent a third frequency range, the fourth set of coefficients 315-d (e.g., fourth component) may represent a fourth frequency range, and the fifth set of coefficients 315-e (e.g., fifth component) may represent a fifth frequency range. Each respective set of coefficients 315 may include one or more coefficients, and may be referred to as “components.” Although depicted as a particular quantity of sets of coefficients 315 for illustrative purposes, the input signal(s) 305 may be decomposed into any quantity and combination of coefficients 315/sets of coefficients 315.

In some cases, one or more of the sets of coefficients 315 may undergo an inverse DWT (IDWT) 320, a noise thresholding procedure 325, or both. For example, the IDWT may remove one or more sets of coefficients 315 associated with a frequency range outside of (e.g., greater than or less than) a threshold frequency range. The threshold frequency range may be an accepted frequency range, where the noise filtering procedure 300 may attempt to remove any sets of coefficients 315 outside of the threshold frequency range.

In some aspects, the noise filtering procedure 300 may be configured to filter, cancel, or otherwise remove frequencies/coefficients 315 that are not typically associated with respective physiological phenomena. As such, the noise filtering procedure 300 may consider coefficients outside of the threshold frequency range to be noise. In some instances, the threshold frequency range(s) may be predetermined (e.g., a universal thresholding rule). In some other instances, the threshold frequency range may be dynamic. A thresholding rule may be difficult to adjust dynamically as the spectral power shifts to different coefficient 315 levels based on an activity or a person. However, the threshold frequency range(s) may be dynamic in that the noise filtering procedure 300 dynamically selects the sets of coefficients 315. For instance, the threshold frequency (e.g., threshold frequency range(s)) may apply a universal threshold rule (e.g., sqtwolog) to predetermined or dynamically selected sets of coefficients 315.

Additionally, or alternatively, the one or more sets of coefficients 315 may undergo a noise thresholding 325 (e.g., the noise filtering procedure 300 may employ the IDWT 320, the noise thresholding 325, or both). The noise filtering procedure 300 may calculate a noise reference as part of a noise thresholding 325. For example, the noise thresholding 325 may collect frequencies (e.g., anomaly frequencies) that have a frequency above a specified frequency threshold. For instance, the noise filtering procedure 300 may calculate the noise reference based on the anomaly frequencies. The anomaly frequencies may be associated with sporadic movements (e.g., hand movement, finger movement, and the like). In some instances, the noise filtering procedure 300 may remove (e.g., filter) one or more characteristics including the noise component based on the noise reference and the noise thresholding 325.

In some cases, the noise filtering procedure may include an adaptive noise cancelation (ANC) 330 algorithm. In some examples, the ANC 330 may receive the noise component and one or more pre-processed signals as inputs, and may generate an output signal 335.

In this regard, the noise filtering procedure 300 may calculate (e.g., result in) an output signal 335. For instance, the wearable device 104-a may calculate a second signal (e.g., the output signal 335) based on filtering the one or more characteristics including the noise component from the first signal(s) (e.g., the input signal(s) 305). In some instances, the output signal 335 is a filtered and cleaner (e.g., more accurate) version of the input signal(s) 305. More specifically, the second signal (e.g., output signal 335) may be associated with a clean signal. As such, the output signal 335 may be used to more accurately and efficiently evaluate physiological characteristics/phenomenon of the user 102 (e.g., heart rate, blood oxygen, respiration rate, blood pressure, HRV, etc.).

In some cases, the noise filtering procedure 300 may include measurements performed over a time window. In some instances, one or more PPG measurements may not be read accurately and may be considered unavailable. This unavailability may be described by one or more unavailability percentage metrics (e.g., unavailability percentage (UAP), big error percentage (BEP), mean absolute error (MAE), mean absolute percentage error (MAPE)).

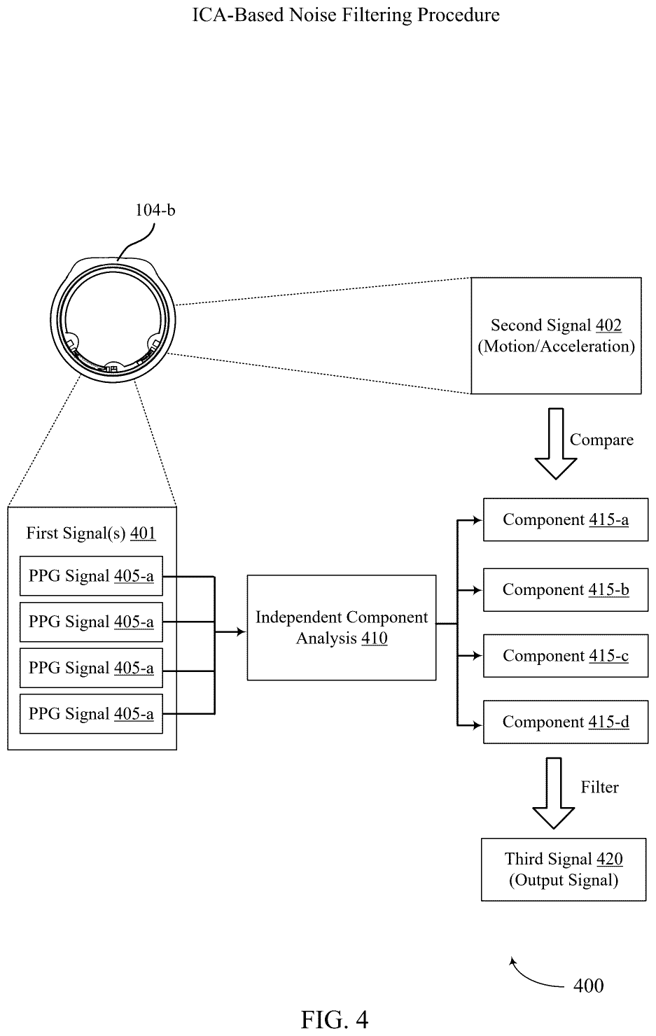

FIG. 4 shows an example of a noise filtering procedure 400 that supports techniques for noise reduction in accordance with aspects of the present disclosure. The noise filtering procedure 400 (e.g., filtering procedure) may implement, or be implemented by, aspects of the system 100, the system 200, the noise filtering procedure 300, or any combination thereof. The noise filtering procedure 300 shown and described in FIG. 3 may be performed at a wearable device 104-a, a user device 106, one or more servers 110, or any combination thereof, as described with reference to FIG. 1 and FIG. 2. The wearable device 104-a may be any example of a wearable ring device 104, a watch or other wrist-worn wearable device (e.g., bracelet), a necklace, and the like.

The noise filtering procedure 400 illustrated in FIG. 4 illustrates an example of a ICA-based noise filtering procedure that may be used to reduce or eliminate noise from signals/measurements performed via a wearable device 104-b. In some cases, the noise filtering procedure 400 may effectively eliminate one or more noise components from signals acquired by the wearable device 104-a, and may result in a clean signal that may be used to more accurately and reliably perform physiological measurements of the user 102 (e.g., heart rate measurements, HRV measurements, blood oxygen measurements, blood pressure measurements, etc.).

For example, a wearable device 104-b may emit light from a set of light emitting elements (e.g., one or more LEDs), and may receive the light reflected/refracted from a tissue of a user 102 using one or more photodetectors. The photodetectors may generate one or more first signals 401 (e.g., PPG signals 405) that are based on the light received via the photodetectors. Each PPG signal 405 (e.g., a PPG signal 405-a, a PPG signal 405-b, a PPG signal 405-c, and a PPG signal 405-d) may include a mixture of PPG data. The first signals 401 (e.g., the respective PPG signals 405) may include a representation of one or more physiological phenomena of the user, such as heart rate measurements, blood oxygen levels measurements, etc. The PPG signals 405 may further include a noise component from the set of light emitting elements and/or photodetectors used to acquire the respective signals. The first signals 401 (e.g., PPG signals 405) may be measured over a specified time window, where the noise component of the input signal 305 may be associated with or attributable to movement (e.g., user motion) during the time interval. For example, movement of the user's hand, finger, etc., as well as movement of the wearable device 104-a relative to the user's tissue (e.g., the ring rotating around the user's finger) may result in noise/motion artifacts within the acquired PPG signals 405.

In some instances, each respective PPG signal 405 may vary based on the time the PPG signals 405 were acquired, the combination of light-emitting and light-receiving components used to acquire the PPG signals 405, the wavelengths of light used to acquire the respective PPG signals 405 (e.g., green, red, or IR light). For example, the PPG signals 405 may vary in signal strength or intensity, phase, etc.

In some cases, a wearable device 104-b, or another device associated with the wearable device 104-b, may measure a second signal 402. The second signal 402 may be collected using an accelerometer, a gyroscope, or some other motion-detecting sensor of the wearable device 104-b. For example, an accelerometer of the wearable device 104-b may measure a motion (e.g., acceleration or force in a direction) or a vibration by converting physical movement into one or more electrical movements. For example, an accelerometer may track (e.g., and the second signal may include) movement and activity of the user 102 (e.g., steps taken, distance traveled, calories burned, and the like).

In some cases, the noise filtering procedure 400 may involve processing the first signal(s) 401. For example, the noise filtering procedure 400 may process the first signal(s) 401 (e.g., PPG signals 405) to filter (e.g., remove or eliminate) one or more characteristics including the noise component. The noise filtering procedure 400 may include an ICA 410 (e.g., ICA-based noise filtering procedure 400). Additionally, or alternatively, the noise filtering procedure 400 may include a singular value decomposition (SVD). The SVD may give a set of singular values that represent components such as pulse waves, motion artifacts, high frequency noise, and the like. These components may be independent or dependent.

For example, the ICA 410 may decompose (e.g., extract) the one or more PPG signals 405 (e.g., one or more channels) into a set of one or more components 415 (e.g., a component 415-a, a component 415-b, a component 415-c, a component 415-d, and a component 415-e). In some instances, each component 415 may be associated with (e.g., represent) an independent component. For instance, the component 415-a may represent a first independent component, the component 415-b may represent a second independent component, the component 415-c may represent a third independent component, and the component 415-d may represent a fourth independent component. Examples of independent components may be attributable to, or associated with, hand movements of the user, finger movements of the user, steps, a heart rate of the user, or the like. Although depicted as a particular quantity of PPG signals 405 and components 415 for illustrative purposes, the noise filtering procedure 400 may include any quantity and combination of PPG signals 405 and components 415.

In some cases, the noise filtering procedure 400 may compare one or more components 415 with the second signal 402. For example, the noise filtering procedure 400 may compare the one or more components 415 with the second signal 402 obtained from the accelerometer of the wearable device 104-b. The noise filtering procedure 400 may calculate a correlation between the one or more components 415 and the second signal 402 (e.g., find which ICA components 415 correlate with the accelerometer). In other words, the noise filtering procedure 400 may compare the components 415 of the PPG signals 405 to determine which components are associated with (e.g., correlated with) the movement of the user/wearable device 104-b within the second signal 402.

In some cases, the noise filtration procedure 400 may determine whether the correlation between each of the one or more components 415 and the second signal 402 is greater than a threshold correlation. As a specific example, a device (e.g., wearable device 104-b, user device 106, servers 110, etc.) may determine that the component 415-a and the component 415-b have a correlation with the second signal 402 that is greater than the threshold correlation, but the component 415-c and the component 415-d may not have a correlation with the second signal 402 that is greater than the threshold correlation.

In some cases, the system (e.g., wearable device 104-b, user device 106, servers 110, etc.) may calculate a noise reference. For example, the system may calculate a noise reference based on each of the one or more components 415 associated with a correlation greater than the threshold correlation. In other words, the system may compose a noise reference using highly correlated components 415. Following the specific example mentioned above, the system may calculate the noise reference based on the component 415-a and the component 415-b (due to the high correlation between the components 415-a, 415-b and the second signal 402). The noise filtering procedure 400 may filter the one or more characteristics including the noise component from the first signal(s) 401 based on this noise reference.

In some cases, the noise filtering procedure 400 may calculate (e.g., result in) a third signal 420 (e.g., filtered output signal). For instance, the wearable device 104-b (and/or the user device 106, servers 110, or any combination thereof) may calculate a third signal 420 based on filtering the one or more characteristics including the noise component from the first signal(s) 401 (e.g., the PPG signals 405) and comparing the one or more components 415 with the second signal 402. In some instances, the third signal 420 may be more accurate than the first signal(s) 401. More specifically, the third signal 420 (e.g., output signal) may be associated with a clean signal. As such, the third signal 420 may be used to more accurately and efficiently evaluate physiological characteristics/phenomenon of the user 102 (e.g., heart rate, blood oxygen, respiration rate, blood pressure, HRV, etc.).

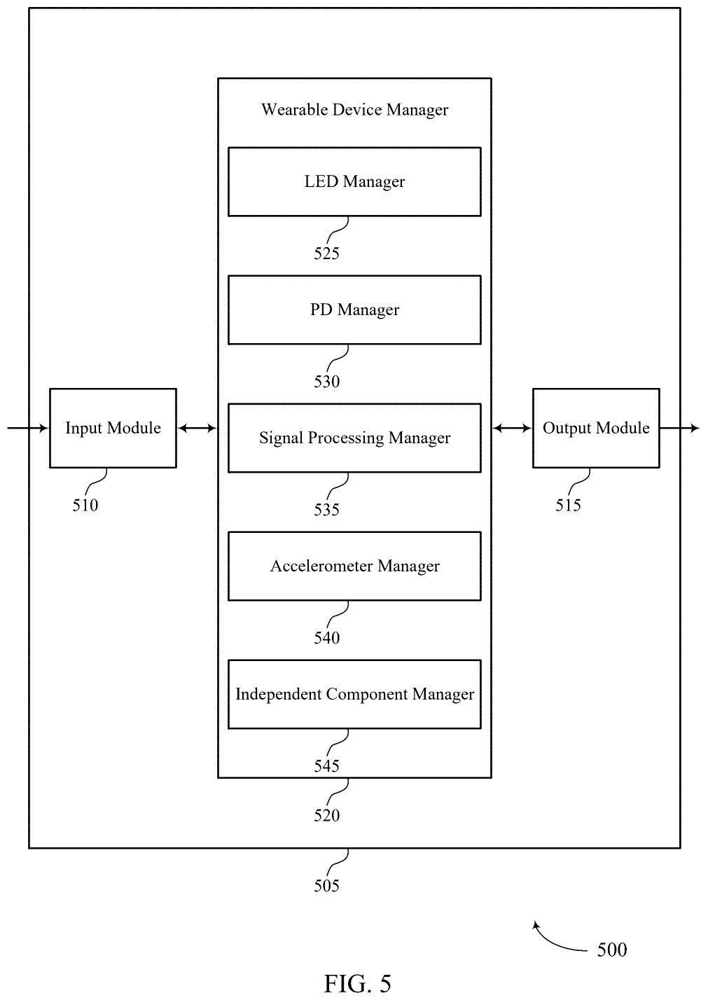

FIG. 5 shows a block diagram 500 of a device 505 that supports techniques for noise reduction in accordance with aspects of the present disclosure. The device 505 may include an input module 510, an output module 515, and a wearable device manager 520. The device 505, or one of more components of the device 505 (e.g., the input module 510, the output module 515, and the wearable device manager 520), may include at least one processor, which may be coupled with at least one memory, to support the described techniques. Each of these components may be in communication with one another (e.g., via one or more buses).

For example, the wearable device manager 520 may include a LED manager 525, a PD manager 530, a signal processing manager 535, an accelerometer manager 540, an independent component manager 545, or any combination thereof. In some examples, the wearable device manager 520, or various components thereof, may be configured to perform various operations (e.g., receiving, monitoring, transmitting) using or otherwise in cooperation with the input module 510, the output module 515, or both. For example, the wearable device manager 520 may receive information from the input module 510, send information to the output module 515, or be integrated in combination with the input module 510, the output module 515, or both to receive information, transmit information, or perform various other operations as described herein.

The wearable device manager 520 may support noise filtering for a wearable ring device in accordance with examples as disclosed herein. The LED manager 525 may be configured as or otherwise support a means for emitting light from a set of light emitting elements of the wearable ring device. The PD manager 530 may be configured as or otherwise support a means for measuring a first signal comprising a representation of one or more physiological phenomenon and a noise component from the set of light emitting elements, wherein the noise component corresponds to a movement associated with the wearable ring device. The signal processing manager 535 may be configured as or otherwise support a means for processing the first signal to filter one or more characteristics including the noise component from the first signal based at least in part on a filtering procedure, wherein the filtering procedure comprises decomposing the first signal into a set of coefficients, and wherein each coefficient is associated with a frequency range. The signal processing manager 535 may be configured as or otherwise support a means for calculating a second signal based at least in part on filtering the one or more characteristics including the noise component from the first signal.

Additionally, or alternatively, the wearable device manager 520 may support noise filtering for a wearable ring device in accordance with examples as disclosed herein. The LED manager 525 may be configured as or otherwise support a means for emitting light from a set of light emitting elements of the wearable ring device. The PD manager 530 may be configured as or otherwise support a means for measuring one or more first signals comprising a representation of one or more physiological phenomenon and one or more noise components from the set of light emitting elements, wherein the one or more noise components correspond to a movement associated with the wearable ring device. The accelerometer manager 540 may be configured as or otherwise support a means for measuring a second signal associated with an accelerometer. The signal processing manager 535 may be configured as or otherwise support a means for processing the one or more first signals to filter one or more characteristics including the one or more noise components from the one or more first signals based at least in part on a filtering procedure, wherein the filtering procedure comprises decomposing the one or more first signals into a set of independent components. The independent component manager 545 may be configured as or otherwise support a means for comparing one or more independent components of the set of independent components with the second signal. The signal processing manager 535 may be configured as or otherwise support a means for calculating a third signal based at least in part on filtering the one or more characteristics including the one or more noise components from the one or more first signals and comparing the one or more independent components with the second signal.

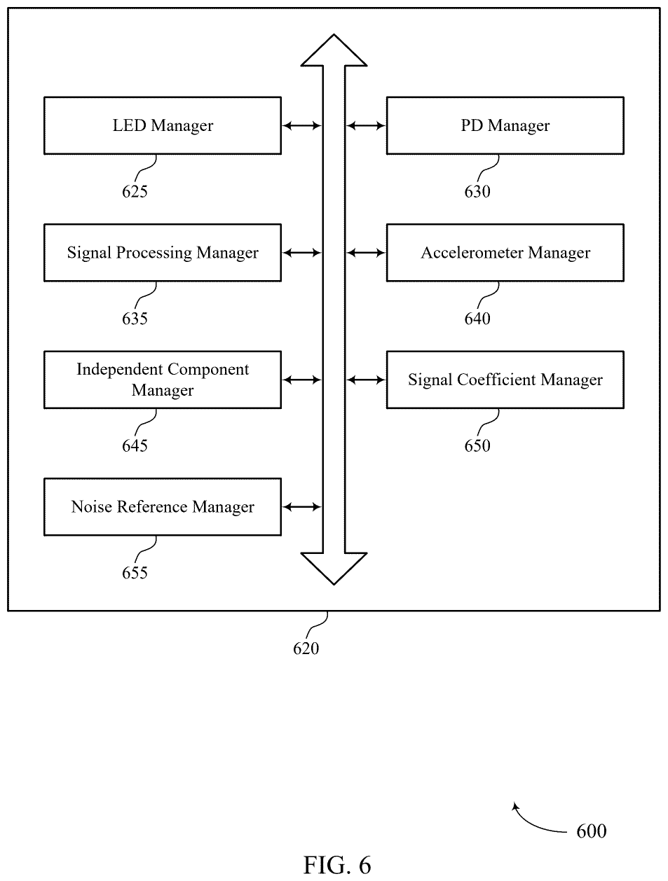

FIG. 6 shows a block diagram 600 of a wearable device manager 620 that supports techniques for noise reduction in accordance with aspects of the present disclosure. The wearable device manager 620 may be an example of aspects of a wearable device manager or a wearable device manager 520, or both, as described herein. The wearable device manager 620, or various components thereof, may be an example of means for performing various aspects of techniques for noise reduction as described herein. For example, the wearable device manager 620 may include a LED manager 625, a PD manager 630, a signal processing manager 635, an accelerometer manager 640, an independent component manager 645, a signal coefficient manager 650, a noise reference manager 655, or any combination thereof. Each of these components, or components of subcomponents thereof (e.g., one or more processors, one or more memories), may communicate, directly or indirectly, with one another (e.g., via one or more buses).

The wearable device manager 620 may support noise filtering for a wearable ring device in accordance with examples as disclosed herein. The LED manager 625 may be configured as or otherwise support a means for emitting light from a set of light emitting elements of the wearable ring device. The PD manager 630 may be configured as or otherwise support a means for measuring a first signal comprising a representation of one or more physiological phenomenon and a noise component from the set of light emitting elements, wherein the noise component corresponds to a movement associated with the wearable ring device. The signal processing manager 635 may be configured as or otherwise support a means for processing the first signal to filter one or more characteristics including the noise component from the first signal based at least in part on a filtering procedure, wherein the filtering procedure comprises decomposing the first signal into a set of coefficients, and wherein each coefficient is associated with a frequency range. In some examples, the signal processing manager 635 may be configured as or otherwise support a means for calculating a second signal based at least in part on filtering the one or more characteristics including the noise component from the first signal.

In some examples, the filtering procedure comprises DWT procedure.

In some examples, to support processing the first signal to filter the one or more characteristics including the noise component, the signal coefficient manager 650 may be configured as or otherwise support a means for removing one or more coefficients of the set of coefficients, wherein the one or more coefficients are associated with a frequency range that falls outside of a threshold frequency range.

In some examples, the noise reference manager 655 may be configured as or otherwise support a means for calculating a noise reference based at least in part on the one or more coefficients, wherein filtering the one or more characteristics including the noise component is based at least in part on the noise reference.

In some examples, the threshold frequency range is predetermined or dynamic.

In some examples, the second signal is associated with a clean signal.

In some examples, the set of light emitting elements comprises one or more LEDs.

In some examples, the one or more physiological phenomenon comprises blood oxygen levels, heart rate measurements, or both.

Additionally, or alternatively, the wearable device manager 620 may support noise filtering for a wearable ring device in accordance with examples as disclosed herein. In some examples, the LED manager 625 may be configured as or otherwise support a means for emitting light from a set of light emitting elements of the wearable ring device. In some examples, the PD manager 630 may be configured as or otherwise support a means for measuring one or more first signals comprising a representation of one or more physiological phenomenon and one or more noise components from the set of light emitting elements, wherein the one or more noise components correspond to a movement associated with the wearable ring device. The accelerometer manager 640 may be configured as or otherwise support a means for measuring a second signal associated with an accelerometer. In some examples, the signal processing manager 635 may be configured as or otherwise support a means for processing the one or more first signals to filter one or more characteristics including the one or more noise components from the one or more first signals based at least in part on a filtering procedure, wherein the filtering procedure comprises decomposing the one or more first signals into a set of independent components. The independent component manager 645 may be configured as or otherwise support a means for comparing one or more independent components of the set of independent components with the second signal. In some examples, the signal processing manager 635 may be configured as or otherwise support a means for calculating a third signal based at least in part on filtering the one or more characteristics including the one or more noise components from the one or more first signals and comparing the one or more independent components with the second signal.

In some examples, the filtering procedure comprises an ICA.

In some examples, to support comparing the one or more independent components with the second signal, the signal processing manager 635 may be configured as or otherwise support a means for calculating a correlation between the one or more independent components and the second signal.

In some examples, the signal processing manager 635 may be configured as or otherwise support a means for determining whether the correlation between each of the one or more independent components and the second signal is greater than a threshold correlation based at least in part on calculating the correlation.

In some examples, the noise reference manager 655 may be configured as or otherwise support a means for calculating a noise reference based at least in part on each of the one or more independent components associated with a correlation greater than the threshold correlation, wherein filtering the one or more characteristics including the one or more noise components from the one or more first signals is based at least in part on the noise reference.

In some examples, the third signal is associated with a clean signal.

In some examples, the set of light emitting elements comprises one or more LEDs.

In some examples, the one or more physiological phenomenon comprises blood oxygen levels, heart rate measurements, or both.

In some examples, the set of independent components comprises at least hand or finger movement, steps, heart rate, or a combination thereof.

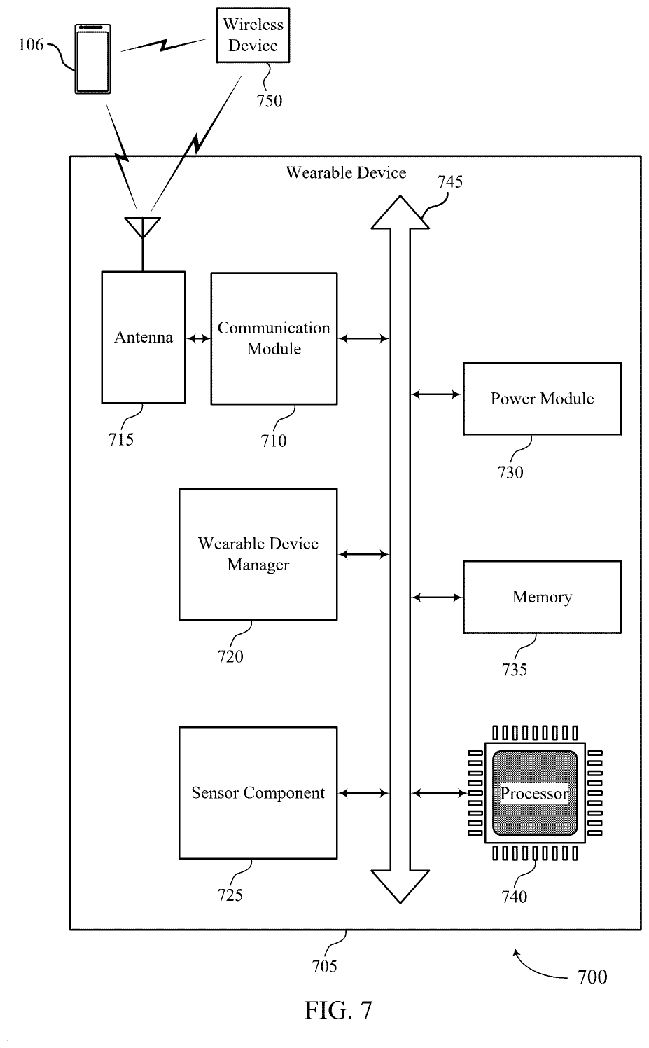

FIG. 7 shows a diagram of a system 700 including a device 705 that supports techniques for noise reduction in accordance with aspects of the present disclosure. The device 705 may be an example of or include the components of a device 505 as described herein. The device 705 may include an example of a wearable device 104, as described previously herein. The device 705 may include components for bi-directional communications including components for transmitting and receiving communications with a user device 106 and a server 110, such as a wearable device manager 720, a communication module 710, an antenna 715, a sensor component 725, a power module 730, at least one memory 735, at least one processor 740, and a wireless device 750. These components may be in electronic communication or otherwise coupled (e.g., operatively, communicatively, functionally, electronically, electrically) via one or more buses (e.g., a bus 745).

The wearable device manager 720 may support noise filtering for a wearable ring device in accordance with examples as disclosed herein. For example, the wearable device manager 720 may be configured as or otherwise support a means for emitting light from a set of light emitting elements of the wearable ring device. The wearable device manager 720 may be configured as or otherwise support a means for measuring a first signal comprising a representation of one or more physiological phenomenon and a noise component from the set of light emitting elements, wherein the noise component corresponds to a movement associated with the wearable ring device. The wearable device manager 720 may be configured as or otherwise support a means for processing the first signal to filter one or more characteristics including the noise component from the first signal based at least in part on a filtering procedure, wherein the filtering procedure comprises decomposing the first signal into a set of coefficients, and wherein each coefficient is associated with a frequency range. The wearable device manager 720 may be configured as or otherwise support a means for calculating a second signal based at least in part on filtering the one or more characteristics including the noise component from the first signal.

Additionally, or alternatively, the wearable device manager 720 may support noise filtering for a wearable ring device in accordance with examples as disclosed herein. For example, the wearable device manager 720 may be configured as or otherwise support a means for emitting light from a set of light emitting elements of the wearable ring device. The wearable device manager 720 may be configured as or otherwise support a means for measuring one or more first signals comprising a representation of one or more physiological phenomenon and one or more noise components from the set of light emitting elements, wherein the one or more noise components correspond to a movement associated with the wearable ring device. The wearable device manager 720 may be configured as or otherwise support a means for measuring a second signal associated with an accelerometer. The wearable device manager 720 may be configured as or otherwise support a means for processing the one or more first signals to filter one or more characteristics including the one or more noise components from the one or more first signals based at least in part on a filtering procedure, wherein the filtering procedure comprises decomposing the one or more first signals into a set of independent components. The wearable device manager 720 may be configured as or otherwise support a means for comparing one or more independent components of the set of independent components with the second signal. The wearable device manager 720 may be configured as or otherwise support a means for calculating a third signal based at least in part on filtering the one or more characteristics including the one or more noise components from the one or more first signals and comparing the one or more independent components with the second signal.

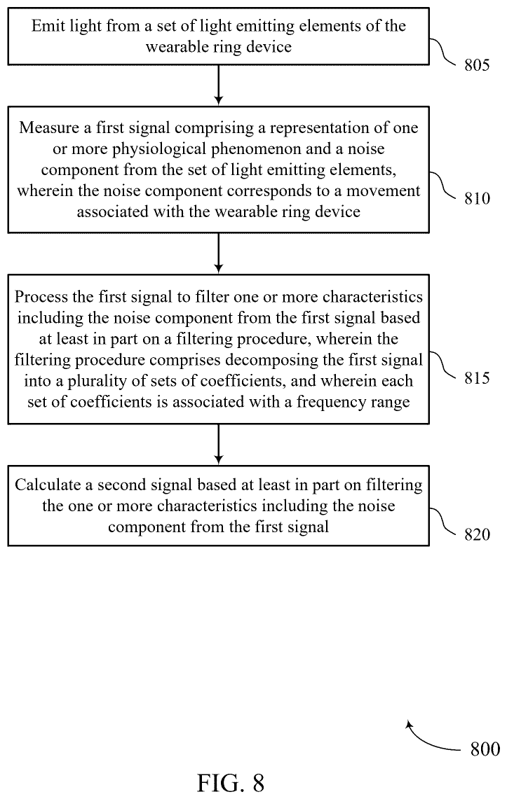

FIG. 8 shows a flowchart illustrating a method 800 that supports techniques for noise reduction in accordance with aspects of the present disclosure. The operations of the method 800 may be implemented by a wearable device or its components as described herein. For example, the operations of the method 800 may be performed by a wearable device as described with reference to FIGS. 1 through 7. In some examples, a wearable device may execute a set of instructions to control the functional elements of the wearable device to perform the described functions. Additionally, or alternatively, the wearable device may perform aspects of the described functions using special-purpose hardware.

At 805, the method may include emitting light from a set of light emitting elements of the wearable ring device. The operations of block 805 may be performed in accordance with examples as disclosed herein. In some examples, aspects of the operations of 805 may be performed by a LED manager 625 as described with reference to FIG. 6.

At 810, the method may include measuring a first signal comprising a representation of one or more physiological phenomenon and a noise component from the set of light emitting elements, wherein the noise component corresponds to a movement associated with the wearable ring device. The operations of block 810 may be performed in accordance with examples as disclosed herein. In some examples, aspects of the operations of 810 may be performed by a PD manager 630 as described with reference to FIG. 6.

At 815, the method may include processing the first signal to filter one or more characteristics including the noise component from the first signal based at least in part on a filtering procedure, wherein the filtering procedure comprises decomposing the first signal into a plurality of sets of coefficients, and wherein each set of coefficients is associated with a frequency range. The operations of block 815 may be performed in accordance with examples as disclosed herein. In some examples, aspects of the operations of 815 may be performed by a signal processing manager 635 as described with reference to FIG. 6.

At 820, the method may include calculating a second signal based at least in part on filtering the one or more characteristics including the noise component from the first signal. The operations of block 820 may be performed in accordance with examples as disclosed herein. In some examples, aspects of the operations of 820 may be performed by a signal processing manager 635 as described with reference to FIG. 6.

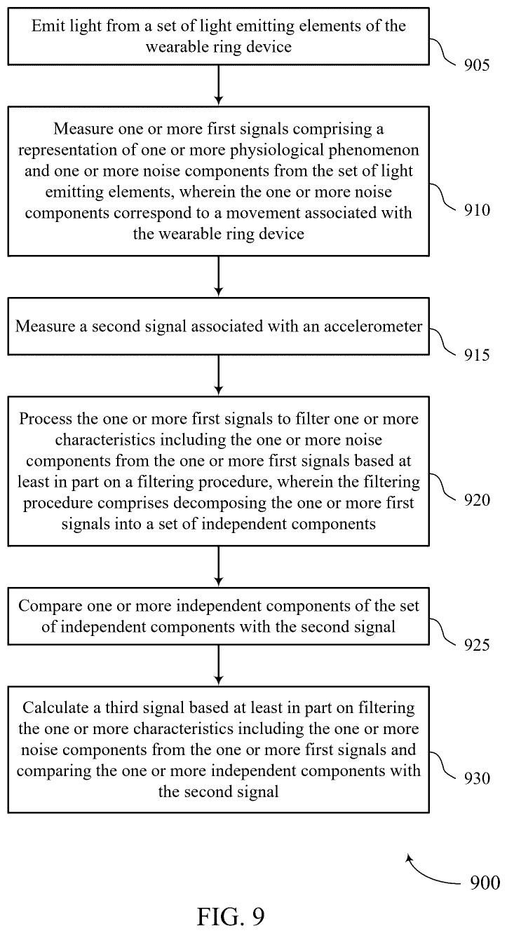

FIG. 9 shows a flowchart illustrating a method 900 that supports techniques for noise reduction in accordance with aspects of the present disclosure. The operations of the method 900 may be implemented by a wearable device or its components as described herein. For example, the operations of the method 900 may be performed by a wearable device as described with reference to FIGS. 1 through 7. In some examples, a wearable device may execute a set of instructions to control the functional elements of the wearable device to perform the described functions. Additionally, or alternatively, the wearable device may perform aspects of the described functions using special-purpose hardware.

At 905, the method may include emitting light from a set of light emitting elements of the wearable ring device. The operations of block 905 may be performed in accordance with examples as disclosed herein. In some examples, aspects of the operations of 905 may be performed by a LED manager 625 as described with reference to FIG. 6.

At 910, the method may include measuring one or more first signals comprising a representation of one or more physiological phenomenon and one or more noise components from the set of light emitting elements, wherein the one or more noise components correspond to a movement associated with the wearable ring device. The operations of block 910 may be performed in accordance with examples as disclosed herein. In some examples, aspects of the operations of 910 may be performed by a PD manager 630 as described with reference to FIG. 6.

At 915, the method may include measuring a second signal associated with an accelerometer. The operations of block 915 may be performed in accordance with examples as disclosed herein. In some examples, aspects of the operations of 915 may be performed by an accelerometer manager 640 as described with reference to FIG. 6.

At 920, the method may include processing the one or more first signals to filter one or more characteristics including the one or more noise components from the one or more first signals based at least in part on a filtering procedure, wherein the filtering procedure comprises decomposing the one or more first signals into a set of independent components. The operations of block 920 may be performed in accordance with examples as disclosed herein. In some examples, aspects of the operations of 920 may be performed by a signal processing manager 635 as described with reference to FIG. 6.

At 925, the method may include comparing one or more independent components of the set of independent components with the second signal. The operations of block 925 may be performed in accordance with examples as disclosed herein. In some examples, aspects of the operations of 925 may be performed by an independent component manager 645 as described with reference to FIG. 6.

At 930, the method may include calculating a third signal based at least in part on filtering the one or more characteristics including the one or more noise components from the one or more first signals and comparing the one or more independent components with the second signal. The operations of block 930 may be performed in accordance with examples as disclosed herein. In some examples, aspects of the operations of 930 may be performed by a signal processing manager 635 as described with reference to FIG. 6.

It should be noted that the methods described above describe possible implementations, and that the operations and the steps may be rearranged or otherwise modified and that other implementations are possible. Furthermore, aspects from two or more of the methods may be combined.

A method for noise filtering for a wearable ring device by an apparatus is described. The method may include emitting light from a set of light emitting elements of the wearable ring device, measuring a first signal comprising a representation of one or more physiological phenomenon and a noise component from the set of light emitting elements, wherein the noise component corresponds to a movement associated with the wearable ring device, processing the first signal to filter one or more characteristics including the noise component from the first signal based at least in part on a filtering procedure, wherein the filtering procedure comprises decomposing the first signal into a set of coefficients, and wherein each coefficient is associated with a frequency range, and calculating a second signal based at least in part on filtering the one or more characteristics including the noise component from the first signal.

An apparatus for noise filtering for a wearable ring device is described. The apparatus may include one or more memories storing processor executable code, and one or more processors coupled with the one or more memories. The one or more processors may individually or collectively operable to execute the code to cause the apparatus to emit light from a set of light emitting elements of the wearable ring device, measure a first signal comprising a representation of one or more physiological phenomenon and a noise component from the set of light emitting elements, wherein the noise component corresponds to a movement associated with the wearable ring device, process the first signal to filter one or more characteristics including the noise component from the first signal based at least in part on a filtering procedure, wherein the filtering procedure comprises decomposing the first signal into a set of coefficients, and wherein each coefficient is associated with a frequency range, and calculate a second signal based at least in part on filtering the one or more characteristics including the noise component from the first signal.

Another apparatus for noise filtering for a wearable ring device is described. The apparatus may include means for emitting light from a set of light emitting elements of the wearable ring device, means for measuring a first signal comprising a representation of one or more physiological phenomenon and a noise component from the set of light emitting elements, wherein the noise component corresponds to a movement associated with the wearable ring device, means for processing the first signal to filter one or more characteristics including the noise component from the first signal based at least in part on a filtering procedure, wherein the filtering procedure comprises decomposing the first signal into a set of coefficients, and wherein each coefficient is associated with a frequency range, and means for calculating a second signal based at least in part on filtering the one or more characteristics including the noise component from the first signal.

A non-transitory computer-readable medium storing code for noise filtering for a wearable ring device is described. The code may include instructions executable by one or more processors to emit light from a set of light emitting elements of the wearable ring device, measure a first signal comprising a representation of one or more physiological phenomenon and a noise component from the set of light emitting elements, wherein the noise component corresponds to a movement associated with the wearable ring device, process the first signal to filter one or more characteristics including the noise component from the first signal based at least in part on a filtering procedure, wherein the filtering procedure comprises decomposing the first signal into a set of coefficients, and wherein each coefficient is associated with a frequency range, and calculate a second signal based at least in part on filtering the one or more characteristics including the noise component from the first signal.

In some examples of the method, apparatus, and non-transitory computer-readable medium described herein, the filtering procedure comprises DWT procedure.

In some examples of the method, apparatus, and non-transitory computer-readable medium described herein, processing the first signal to filter the one or more characteristics including the noise component may include operations, features, means, or instructions for removing one or more coefficients of the set of coefficients, wherein the one or more coefficients may be associated with a frequency range that falls outside of a threshold frequency range.

Some examples of the method, apparatus, and non-transitory computer-readable medium described herein may further include operations, features, means, or instructions for calculating a noise reference based at least in part on the one or more coefficients, wherein filtering the one or more characteristics including the noise component may be based at least in part on the noise reference.

In some examples of the method, apparatus, and non-transitory computer-readable medium described herein, the threshold frequency range may be predetermined or dynamic.

In some examples of the method, apparatus, and non-transitory computer-readable medium described herein, the second signal may be associated with a clean signal.

In some examples of the method, apparatus, and non-transitory computer-readable medium described herein, the set of light emitting elements comprises one or more LEDs.

In some examples of the method, apparatus, and non-transitory computer-readable medium described herein, the one or more physiological phenomenon comprises blood oxygen levels, heart rate measurements, or both.

A method for noise filtering for a wearable ring device by an apparatus is described. The method may include emitting light from a set of light emitting elements of the wearable ring device, measuring one or more first signals comprising a representation of one or more physiological phenomenon and one or more noise components from the set of light emitting elements, wherein the one or more noise components correspond to a movement associated with the wearable ring device, measuring a second signal associated with an accelerometer, processing the one or more first signals to filter one or more characteristics including the one or more noise components from the one or more first signals based at least in part on a filtering procedure, wherein the filtering procedure comprises decomposing the one or more first signals into a set of independent components, comparing one or more independent components of the set of independent components with the second signal, and calculating a third signal based at least in part on filtering the one or more characteristics including the one or more noise components from the one or more first signals and comparing the one or more independent components with the second signal.

An apparatus for noise filtering for a wearable ring device is described. The apparatus may include one or more memories storing processor executable code, and one or more processors coupled with the one or more memories. The one or more processors may individually or collectively operable to execute the code to cause the apparatus to emit light from a set of light emitting elements of the wearable ring device, measure one or more first signals comprising a representation of one or more physiological phenomenon and one or more noise components from the set of light emitting elements, wherein the one or more noise components correspond to a movement associated with the wearable ring device, measure a second signal associated with an accelerometer, process the one or more first signals to filter one or more characteristics including the one or more noise components from the one or more first signals based at least in part on a filtering procedure, wherein the filtering procedure comprises decomposing the one or more first signals into a set of independent components, compare one or more independent components of the set of independent components with the second signal, and calculate a third signal based at least in part on filtering the one or more characteristics including the one or more noise components from the one or more first signals and comparing the one or more independent components with the second signal.

Another apparatus for noise filtering for a wearable ring device is described. The apparatus may include means for emitting light from a set of light emitting elements of the wearable ring device, means for measuring one or more first signals comprising a representation of one or more physiological phenomenon and one or more noise components from the set of light emitting elements, wherein the one or more noise components correspond to a movement associated with the wearable ring device, means for measuring a second signal associated with an accelerometer, means for processing the one or more first signals to filter one or more characteristics including the one or more noise components from the one or more first signals based at least in part on a filtering procedure, wherein the filtering procedure comprises decomposing the one or more first signals into a set of independent components, means for comparing one or more independent components of the set of independent components with the second signal, and means for calculating a third signal based at least in part on filtering the one or more characteristics including the one or more noise components from the one or more first signals and comparing the one or more independent components with the second signal.

A non-transitory computer-readable medium storing code for noise filtering for a wearable ring device is described. The code may include instructions executable by one or more processors to emit light from a set of light emitting elements of the wearable ring device, measure one or more first signals comprising a representation of one or more physiological phenomenon and one or more noise components from the set of light emitting elements, wherein the one or more noise components correspond to a movement associated with the wearable ring device, measure a second signal associated with an accelerometer, process the one or more first signals to filter one or more characteristics including the one or more noise components from the one or more first signals based at least in part on a filtering procedure, wherein the filtering procedure comprises decomposing the one or more first signals into a set of independent components, compare one or more independent components of the set of independent components with the second signal, and calculate a third signal based at least in part on filtering the one or more characteristics including the one or more noise components from the one or more first signals and comparing the one or more independent components with the second signal.

In some examples of the method, apparatus, and non-transitory computer-readable medium described herein, the filtering procedure comprises an ICA.

In some examples of the method, apparatus, and non-transitory computer-readable medium described herein, comparing the one or more independent components with the second signal may include operations, features, means, or instructions for calculating a correlation between the one or more independent components and the second signal.

Some examples of the method, apparatus, and non-transitory computer-readable medium described herein may further include operations, features, means, or instructions for determining whether the correlation between each of the one or more independent components and the second signal may be greater than a threshold correlation based at least in part on calculating the correlation.

Some examples of the method, apparatus, and non-transitory computer-readable medium described herein may further include operations, features, means, or instructions for calculating a noise reference based at least in part on each of the one or more independent components associated with a correlation greater than the threshold correlation, wherein filtering the one or more characteristics including the one or more noise components from the one or more first signals may be based at least in part on the noise reference.

In some examples of the method, apparatus, and non-transitory computer-readable medium described herein, the third signal may be associated with a clean signal.

In some examples of the method, apparatus, and non-transitory computer-readable medium described herein, the set of light emitting elements comprises one or more LEDs.

In some examples of the method, apparatus, and non-transitory computer-readable medium described herein, the one or more physiological phenomenon comprises blood oxygen levels, heart rate measurements, or both.

In some examples of the method, apparatus, and non-transitory computer-readable medium described herein, the set of independent components comprises at least hand or finger movement, steps, heart rate, or a combination thereof.

The description set forth herein, in connection with the appended drawings, describes example configurations and does not represent all the examples that may be implemented or that are within the scope of the claims. The term “exemplary” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

In the appended figures, similar components or features may have the same reference label. Further, various components of the same type may be distinguished by following the reference label by a dash and a second label that distinguishes among the similar components. If just the first reference label is used in the specification, the description is applicable to any one of the similar components having the same first reference label irrespective of the second reference label.

Information and signals described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative blocks and modules described in connection with the disclosure herein may be implemented or performed with a general-purpose processor, a DSP, an ASIC, an FPGA or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration).

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope of the disclosure and appended claims. For example, due to the nature of software, functions described above can be implemented using software executed by a processor, hardware, firmware, hardwiring, or combinations of any of these. Features implementing functions may also be physically located at various positions, including being distributed such that portions of functions are implemented at different physical locations. Also, as used herein, including in the claims, “or” as used in a list of items (for example, a list of items prefaced by a phrase such as “at least one of” or “one or more of”) indicates an inclusive list such that, for example, a list of at least one of A, B, or C means A or B or C or AB or AC or BC or ABC (i.e., A and B and C). Also, as used herein, the phrase “based on” shall not be construed as a reference to a closed set of conditions. For example, an exemplary step that is described as “based on condition A” may be based on both a condition A and a condition B without departing from the scope of the present disclosure. In other words, as used herein, the phrase “based on” shall be construed in the same manner as the phrase “based at least in part on.”

Computer-readable media includes both non-transitory computer storage media and communication media including any medium that facilitates transfer of a computer program from one place to another. A non-transitory storage medium may be any available medium that can be accessed by a general purpose or special purpose computer. By way of example, and not limitation, non-transitory computer-readable media can comprise RAM, ROM, electrically erasable programmable ROM (EEPROM), compact disk (CD) ROM or other optical disk storage, magnetic disk storage or other magnetic storage devices, or any other non-transitory medium that can be used to carry or store desired program code means in the form of instructions or data structures and that can be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, include CD, laser disc, optical disc, digital versatile disc (DVD), floppy disk and Blu-ray disc where disks usually reproduce data magnetically, while discs reproduce data optically with lasers. Combinations of the above are also included within the scope of computer-readable media.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not limited to the examples and designs described herein, but is to be accorded the broadest scope consistent with the principles and novel features disclosed herein.

What is claimed is:

1. A method for noise filtering for a wearable ring device, comprising:
emitting light from a set of light emitting elements of the wearable ring device;
measuring a first signal comprising a representation of one or more physiological phenomenon and a noise component from the set of light emitting elements, wherein the noise component corresponds to a movement associated with the wearable ring device;
processing the first signal to filter one or more characteristics including the noise component from the first signal based at least in part on a filtering procedure, wherein the filtering procedure comprises decomposing the first signal into a plurality of sets of coefficients, and wherein each set of coefficients is associated with a frequency range; and
calculating a second signal based at least in part on filtering the one or more characteristics including the noise component from the first signal.

2. The method of claim 1, wherein the filtering procedure comprises discrete wavelet transform procedure.

3. The method of claim 1, wherein processing the first signal to filter the one or more characteristics including the noise component further comprises:
removing one or more sets of coefficients of the plurality of sets of coefficients, wherein the one or more sets of coefficients are associated with a frequency range that falls outside of a threshold frequency range.

4. The method of claim 3, further comprising:
calculating a noise reference based at least in part on the one or more sets of coefficients, wherein filtering the one or more characteristics including the noise component is based at least in part on the noise reference.

5. The method of claim 3, wherein the threshold frequency range is predetermined or dynamic.

6. The method of claim 1, wherein the second signal is associated with a clean signal.

7. The method of claim 1, wherein the set of light emitting elements comprises one or more light emitting diodes (LEDs).

8. The method of claim 1, wherein the one or more physiological phenomena comprise blood oxygen levels, heart rate measurements, or both.

9. A method for noise filtering for a wearable ring device, comprising:
emitting light from a set of light emitting elements of the wearable ring device;
measuring one or more first signals comprising a representation of one or more physiological phenomenon and one or more noise components from the set of light emitting elements, wherein the one or more noise components correspond to a movement associated with the wearable ring device;
measuring a second signal associated with an accelerometer;
processing the one or more first signals to filter one or more characteristics including the one or more noise components from the one or more first signals based at least in part on a filtering procedure, wherein the filtering procedure comprises decomposing the one or more first signals into a set of independent components;
comparing one or more independent components of the set of independent components with the second signal; and
calculating a third signal based at least in part on filtering the one or more characteristics including the one or more noise components from the one or more first signals and comparing the one or more independent components with the second signal.

10. The method of claim 9, wherein the filtering procedure comprises an independent component analysis.

11. The method of claim 9, wherein comparing the one or more independent components with the second signal further comprises:
calculating a correlation between the one or more independent components and the second signal.

12. The method of claim 11, further comprising:
determining whether the correlation between each of the one or more independent components and the second signal is greater than a threshold correlation based at least in part on calculating the correlation.

13. The method of claim 12, further comprising:
calculating a noise reference based at least in part on each of the one or more independent components associated with a correlation greater than the threshold correlation, wherein filtering the one or more characteristics including the one or more noise components from the one or more first signals is based at least in part on the noise reference.

14. The method of claim 9, wherein the third signal is associated with a clean signal.

15. The method of claim 9, wherein the set of light emitting elements comprises one or more light emitting diodes (LEDs).

16. The method of claim 9, wherein the one or more physiological phenomenon comprises blood oxygen levels, heart rate measurements, or both.

17. The method of claim 9, wherein the set of independent components comprises at least hand or finger movement, steps, heart rate, or a combination thereof.

18. An apparatus for noise filtering for a wearable ring device, comprising:
one or more memories storing processor-executable code; and
one or more processors coupled with the one or more memories and individually or collectively operable to execute the code to cause the apparatus to:
emit light from a set of light emitting elements of the wearable ring device;
measure a first signal comprising a representation of one or more physiological phenomenon and a noise component from the set of light emitting elements, wherein the noise component corresponds to a movement associated with the wearable ring device;
process the first signal to filter one or more characteristics including the noise component from the first signal based at least in part on a filtering procedure, wherein the filtering procedure comprises decomposing the first signal into a plurality of sets of coefficients, and wherein each set of coefficients is associated with a frequency range; and
calculate a second signal based at least in part on filtering the one or more characteristics including the noise component from the first signal.

19. The apparatus of claim 18, wherein the filtering procedure comprises discrete wavelet transform procedure.

20. The apparatus of claim 18, wherein, to process the first signal to filter the one or more characteristics including the noise component, the one or more processors are individually or collectively further operable to execute the code to cause the apparatus to:
remove one or more sets of coefficients of the plurality of sets of coefficients, wherein the one or more sets of coefficients are associated with a frequency range that falls outside of a threshold frequency range.
